(19) United States
(12) Patent Application Publication
(10) Pub. No.: US 2024/0376480 A1
(43) Pub. Date: Nov. 14, 2024

(54) TREATMENT OF ELEVATED METABOLITE LEVELS USING NANOCARRIERS

(71) Applicant: RAMOT AT TEL-AVIV UNIVERSITY LTD., Tel Aviv (IL)

(72) Inventors: Shai ZILBERZWIGE-TAL, Tel Aviv (IL); Danielle GAZIT, Tel Aviv (IL); Ehud GAZIT, Tel Aviv (IL)

(73) Assignee: RAMOT AT TEL-AVIV UNIVERSITY LTD., Tel Aviv (IL)

(21) Appl. No.: 18/692,766

(22) PCT Filed: Sep. 29, 2022
(86) PCT No.: PCT/IL2022/051035

§ 371 Date: Mar. 15, 2024

Related U.S. Application Data
(60) Provisional application No. 63/249,834, filed on Sep. 29, 2021.
(60) Provisional application No. 63/337,734, filed on May 3, 2022.

(51) Int. Cl. C12N 15/115; A23L 5/20; A23L 13/00; A61K 9/51; A61P 3/00; A61P 19/06; C12N 7/00

(52) U.S. Cl. CPC C12N 15/115; A23L 5/27; A23L 13/00; A61K 9/5184; A61P 3/00; A61P 19/06; C12N 7/00; C12N 2310/16; C12N 2795/18023

(57) ABSTRACT

A novel nano-carrier system and method include a virus-like particle, and a functional RNA encapsulated inside the virus-like particle. The virus-like particle protects the functional RNA from nuclease degradation in vivo, and the functional RNA maintains its functionality while being encapsulated inside the virus-like particle. The system and method may be used to modify, reduce, or treat a disease in a subject.

### FIELD OF THE INVENTION

The present invention relates to RNA nanotechnology, and more particularly, to a system and method utilizing virus-like particles encapsulating functional RNA nanostructures, such as RNA riboswitches. The present invention further relates to administering the nanostructures to a subject to modify, reduce, or treat a disease in the subject. The system further relates to virus-like particles being porous carriers utilized for carrying functional RNA to a target, as well as treating a disease.

### BACKGROUND OF THE INVENTION

A plethora of degenerative disorders, including Alzheimer's disease, Parkinson's disease, and type II diabetes, are all associated with the formation of well-ordered, self-assembled amyloid fibrils. Dobson and co-workers' pioneering work revealed that proteins not related to disease, such as myoglobin, could also form typical amyloid structures. It was shown that apoptotic cell death induction by amyloid assemblies appeared to be a generic property of the formed structures rather than a disease-specific pathology, implying a common mechanism for the toxic effect.

It has also been shown that the generic amyloid hypothesis could be extended to non-proteinaceous metabolite assemblies, such as single amino acids and nucleobases, which also induce apoptotic cell death. The formation of amyloid-like assemblies by metabolites, e.g., adenine, tyrosine, and phenylalanine, implies a general phenomenon of amyloid formation, not limited to proteins and peptides, and offers a new paradigm for metabolic disorders. Metabolic disorders result from the cellular machinery's inability to perform critical biochemical reactions that involve different biosynthetic pathways. In the case of inborn error of metabolism, most disorders (also referred to as IEM disorders) are due to a genetic mutation leading to the malfunction of a single gene typically encoding an enzyme crucial for the metabolic processes. To date, over 50 metabolic disorders have been reported and described, although their relative rarity in the general population resulted in a limited amount of targeted research. However, collectively, metabolic disorders constitute a very substantial part of pediatric genetic diseases. Because the molecular basis of tissue damage is poorly understood, it leaves the patients without disease-modifying treatment.

RNA nanotechnology is a leading field of in vitro molecular-scale device engineering, utilizing the properties of RNA to build architectures such as RNA nanoparticles with nanomedicine applications. Other structural (noncoding) short RNA sequences such as RNA aptamers and short hairpin RNA (shRNA) can be further utilized for medical and technological applications. However, while RNA nanostructures' function is robust under in vitro settings, their implementation as therapeutics and realizing their tremendous potential in gene therapy and targeted drug delivery, as well as other medical and technological uses, requires overcoming their rapid degradation and subsequent loss of function under cellular and serum conditions.

This problem is particularly prominent for RNA-based active ingredients compared to other forms of nucleic acid agents due to the particularly labile nature of RNA molecules, attributed inter alia to their rapid oxidation, the presence of hydroxyl groups on the ribose ring resulting in susceptibility to hydrolysis, spontaneous cleavage of the phosphodiester linkage which may be catalyzed by a variety of agents, and the ubiquity and robust nature of ribonucleases (RNases) in cells and biological fluids. Various approaches have been suggested for enhancing the stability of RNA products, including packaging by liposomes or various proteinaceous coats. However, the applicability of a specific protection method to a specific RNA cargo in the context of a specific intended use remains to be determined, as some of these methods were associated with technical difficulties rendering them impractical for large-scale production and/or for in vivo use (He et al., 2018, ACS Biomater. Sci. Eng., 4, 5, 1708-15).

Viruses are sophisticated supramolecular assemblies, able to protect their nucleic acid content in inhospitable biological environments. Wild-type (WT) MS2 bacteriophage is a 27 nm particle consisting of a single copy of the maturation protein and 180 copies of the coat proteins (CP, organized as 90 homodimers) arranged into an icosahedral shell. The bacteriophage's assembly is driven by a specific 20 nt RNA sequence forming a stem-loop structure called “translational repression RNA” (TR-RNA) encoded in the MS2 genome. The maturation gene is considered to be important for the functionality of the MS2 capsid. A method of forming VLPs in vivo by expressing MS2 CP in E. coli alongside RNA conjugated to the TR-RNA sequence is reviewed in Mikel et al., (2015, Food Environ Virol., 7(2): 96-111).

Virus-like particles (VLPs) are derived in part from viruses through the expression of certain viral structural proteins which make up the viral capsid. This term is often used to describe VLPs that do not contain the viral genome and are non-infectious (although there are some exceptions, for example when describing VLP libraries used for screening). Various viral vectors and VLPs, including those derived from or based on MS2 bacteriophage, have been examined for their potential use as research tools and in the development of potential therapies.

US 2013/0167267 relates to methods and compositions using viral capsids as nanocontainers for producing, isolating and purifying heterologous nucleic acids and proteins. WO 2008/024427 is directed to a library of VLPs of an RNA bacteriophage, where each VLP comprises a coat polypeptide of the phage, modified by insertion of a respective heterologous peptide that is displayed on the VLP. Each VLP encapsidates an mRNA encoding for the respective bacteriophage-VLP. The publication is further directed to VLPs that encapsidate heterologous substances, and their uses, e.g., recognition of potential vaccines. WO 2016/183022 discloses a VLP comprising a capsid enclosing at least one heterologous cargo molecule and at least one packing sequence, wherein the heterologous cargo molecule comprises at least two RNA sequences one of which is the reverse complement of the other and each is longer than 35 nucleotides.

Li et al., (2014, Intern Journal Canc., 134(70): 1683-94), describes applying MS2 VLPs as a delivery vector for mRNA vaccines against prostate cancer. The mRNAs are packaged into the VLPs and delivered thereby into the target cells where they are translated into the immunogenic protein. Similarly, Ashley et al., (2011, ACS Nano, 5, 5729-45), reports the use of MS2 VLPs to selectively deliver nanoparticles, chemotherapeutic drugs, siRNA cocktails, and protein toxins to human hepatocellular carcinoma. The potential use of MS2 VLPs as a delivery platform is reviewed in Fu et al., (2016 Virus Res., 211: 9-16).

Thus, several approaches for delivering RNA agents such as mRNAs encoding a protein of interest and siRNAs directed to a particular gene transcript in a target cell, have been suggested. In order words, hitherto reported methods require disassembly of the VLP capsid and release of the encapsulated RNA cargo in order for said RNA to exert its intended functionality.

Zilberzwige-Tal et al. (2021, ACS Synth. Biol., 10, 1798-1807), to the present inventors and co-workers, discloses encapsulation and protection of functional DNA nanostructures inside MS2 VLPs. Zilberzwige-Tal et al. (2022, ACS Nano, 16, 11733-11741) to the inventors and co-workers, published after the priority dates of the present application, relates to engineered riboswitch nanocarriers as a possible disease-modifying treatment for metabolic disorders.

RNA riboswitches are elements commonly found in the 5′-untranslated region of bacterial mRNAs that are involved in controlling the translation of the respective mRNA transcript, the control being regulated by directly binding a small molecule ligand. These RNA receptors must discriminate between chemically-related metabolites with high selectivity to elicit the appropriate regulatory response. This high-selectivity binding of the riboswitches holds promising potential uses in various fields.

The typical riboswitch contains two distinct functional domains, namely the ligand binding pocket (adopting a compact three-dimensional fold) and the expression platform, which contains a secondary structural switch that interfaces with the transcriptional or translational machinery. Regulation is achieved by virtue of a region of overlap between these two domains, known as the switching sequence, whose pairing directs folding of the RNA into one of two mutually exclusive structures in the expression platform, which represent the on and off states (Garst et al., Cold Spring Harb Perspect Biol 2011; 3:a003533).

Despite considerable research on VLP systems, including bacteriophage VLPs such as MS2, their use in connection with functional RNA cargos having a high degree of structural complexity, including in particular intricate RNA nanostructures such as riboswitches, has not been implemented and characterized. In particular, the ability to produce a nano-carrier system capable of facilitating two mutually exclusive structures of a single RNA molecule, as well as a functional interchange between these structures, while remaining encapsulated within a capsid shell, has not been determined.

There remains a need in the art for nano-carrier systems amenable for maintaining the integrity, stability and activity of functional RNA molecules having high structural complexity under various commercial and clinical settings in vitro and in vivo. There is further a need for effective nano-tools which can selectively eliminate harmful molecules from diverse environments, such as excess metabolites in conditions of metabolic disorders. The development of effective therapeutic agents for the treatment of conditions associated with the formation of metabolite amyloids such as inborn error of metabolism (IEM) disorders, which are amenable for large-scale production while maintaining adequate functionality, efficacy, stability and safety, would be highly advantageous.

### SUMMARY OF THE INVENTION

The invention relates to RNA nanotechnology, in particular to nano-carrier systems comprising a functional RNA encapsulated inside a virus-like particle (VLP), compositions comprising the systems and methods of using same for therapeutic and industrial applications. In particular, the invention in embodiments thereof relates to encapsulation of RNA riboswitches that retain their ability to bind their metabolite ligand and related functionality while being encapsulated inside the VLP, and are protected from nuclease degradation in vivo. Embodiments of the invention further relate to methods of treating and preventing metabolite accumulation in vitro and in vivo, and for the treatment of disorders associated with metabolite toxicity, accumulation and/or aggregation.

The invention is based, in part, on the development of a nano-carrier system, comprising a functional RNA encapsulated inside MS2 bacteriophage VLPs. In particular, disclosed is the construction of a functional RNA in the form of an RNA riboswitch conjugated to a TR-RNA sequence, and co-expression of said functional RNA with an Enterobacteria phage MS2 (MS2) coat proteins (CP) in vivo, in a genetically engineered E. coli system. The co-expression was found to result in self-assembly of the VLP nano-carriers within the living bacteria at high yields; the resulting VLPs were isolated and characterized in various experimental systems and in silico analyses.

As disclosed herein, the VLPs were found to exert exceptional protection of the functional RNA against endonucleases in vivo, and to provide a highly effective protective shell for long-term maintenance of the encapsulated functional riboswitches, which were protected from nuclease degradation and remained functional for over nine months under conventional refrigeration storage. In addition, the riboswitch was surprisingly found to maintain its functionality while being encapsulated inside said VLP. In particular, the encapsulated riboswitch was able to rescue APT1 and AAH1-deficient yeast when supplemented to the culture medium in an excess-adenine yeast model. Remarkably, the nano-carrier system enabled the chelation of excess metabolites, inhibition of their accumulation at toxic levels and formation of metabolite assemblies and amyloids in various environments including in living cells, demonstrating their applicability as a disease-modifying treatment.

These functions were found to be associated with the ability of the encapsulated RNA to retain the structural conformations of both the TR-RNA and the riboswitch under the experimental test conditions, which were further found to be maintained under human physiological conditions.

Accordingly, the invention is further based, in part, on the development of various functional RNA constructs characterized by advantageous structural properties demonstrated to be amenable for maintaining RNA functionality in vivo.

Accordingly, the invention in embodiments thereof relates to a nano-carrier system, comprising a functional RNA encapsulated inside a VLP.

In one aspect, there is provided a nano-carrier system, comprising a functional RNA encapsulated inside MS2 VLP, wherein the functional RNA comprises an RNA riboswitch and a packing sequence, and said riboswitch maintains its functionality while being encapsulated inside said VLP and is protected from nuclease degradation in vivo.

For example, without limitation, the RNA riboswitch may be e.g. a purine riboswitch, a cobalamin riboswitch, a fluoride riboswitch, a glutamine riboswitch, a glycine riboswitch or a xanthine riboswitch (e.g. xanthine riboswitch I). According to some embodiments, the purine riboswitch may include, but is not limited to, adenine riboswitch or guanine riboswitch. In a particular embodiment, said riboswitch is an adenine riboswitch. According to some embodiments the adenine riboswitch may include, but is not limited to, pbuE adenine riboswitch, or add adenine riboswitch. According to some embodiments the guanine riboswitch may include xpt guanine riboswitch. According to some further embodiments the cobalamin riboswitch may include, but is not limited to, Class I cobalamin (Cbi-I) riboswitch or Class II cobalamin (Cbi-II) riboswitch. According to some embodiments, Cbi-II riboswitch may include Cbi-IIa riboswitch or Cbi-IIb riboswitch. Each possibility represents a separate embodiment of the invention.

According to some embodiments the RNA riboswitch may be a naturally occurring riboswitch and/or a synthetically modified riboswitch. According to some embodiments the synthetically modified riboswitch may include one or more nucleotide substitutions (interchangeably referred to herein as “point mutations”) for enhancing the affinity of the riboswitch to at least one ligand. In a particular embodiment said riboswitch is a naturally-occurring or synthetic adenine riboswitch. For example, according to some embodiments, the pbuE adenine riboswitch may include substitution of a uracil nucleotide for the adenine at position 58 of the riboswitch (“A58”), the modified riboswitch designated “A58U”. The nucleic acid sequence of the pbuE A58U adenine riboswitch is set forth in SEQ ID NO: 1, as set forth in Example 1.

Typically and advantageously, the packaging sequence comprises at least one a translational repression RNA (TR-RNA). In another embodiment said packaging sequence is a TR-RNA. In one embodiment said packaging sequence is a MS2 TR-RNA. In another particular embodiment said packaging sequence is a wild-type (WT) MS2 TR-RNA. The nucleic acid sequence of WT MS2 TR-RNA is set forth in SEQ ID NO: 2, as set forth in Example 1.

As disclosed herein, the riboswitch maintains its functionality while being encapsulated inside said VLP, including in particular its specific ligand-binding capacity. As further disclosed herein, this capacity is facilitated by or associated with the formation of a predetermined secondary and/or tertiary conformation, as will be discussed in greater detail below. As is further described herein, functional binding of a riboswitch to its metabolite ligand leads to a conformational change in the riboswitch molecule, such that it assumes a structurally distinct conformation.

Typically, the functional RNA of the invention is characterized in that the riboswitch is located 5′ to the TR-RNA. In one embodiment, said riboswitch and TR-RNA are separated by a non-coding RNA spacer. Typically, the RNA spacer is longer than about 50 nucleotides (nt) in length, more typically longer than 80, 100 or 120 nt in length. In some embodiments, said spacer is up to about 300 nt in length, or in other embodiments up to about 250 or 200 nt in length. In another embodiment said RNA spacer is characterized by a length of 100-200 nt in length, e.g. 100-170, 120-170, 120-200, or 130-160 nt in length. In another embodiment said spacer is characterized by a guanine-cytosine content (GC content) of 20%-70%, e.g. 30%-70%, 20-65%, 30%-65%, 40-65%, or 40%-60%. In another embodiment, said riboswitch is located less than 50, 40, 30 or 20 nt from the 5′ end. Each possibility represents a separate embodiment of the invention.

In an exemplary embodiment, the riboswitch is located 5′ to the TR-RNA, and said riboswitch and TR-RNA are separated by a non-coding RNA spacer characterized by a length of 100-200 nt and a GC content of 20%-70%. In another exemplary embodiment, said riboswitch is located less than 30 nt from the 5′ end, said TR-RNA is located at the 3′ end, and said spacer is 120-170 nt in length and has a GC content of 40%-65%. In another embodiment, said functional RNA has a nucleic acid sequence selected from the group consisting of SEQ ID NOs: 6, 7, 9-13, 19-21 and 23, as set forth in Example 5.

As is further demonstrated herein, the encapsulated riboswitch is protected from nuclease degradation in vivo. Thus, said riboswitch, when encapsulated, remains substantially intact and active under physiological conditions characterized by high nuclease concentrations for a time duration sufficient to exert a therapeutic effect, as will be discussed in greater detail below.

In another embodiment, said VLP comprises a capsid comprising wild-type (WT) MS2 coat proteins (CP). In one embodiment, the MS2 VLP is a WT MS2 VLP. In another embodiment said WT MS2 CP is in the form of a single-chain CP dimer. In another embodiment the CP dimer comprises a linker between the CP monomer units. In another embodiment, said CP or CP dimer further comprises at least one peptide tag (e.g. a histidine tag or an HA tag). In a particular embodiment, said WT MS2 CP is in the form of a single-chain CP dimer optionally comprising a linker between the CP monomer units, and said VLP is devoid of exogenously added targeting motifs.

In another aspect there is provided a nano-carrier system, comprising a riboswitch encapsulated inside a MS2 VLP. In another embodiment there is provided a nano-carrier system, comprising an adenine riboswitch encapsulated inside a MS2 VLP (e.g. WT MS2 VLP).

In another aspect, there is provided a nucleic acid construct encoding the nano-carrier system. In another embodiment the nucleic acid construct is in the form of a prokaryotic or eukaryotic expression vector. For example, without limitation, said construct may be in the form of a plasmid comprising nucleic acid sequences encoding for the functional RNA, the CP and an MS2 maturase protein, operatively link to one or more transcription-regulating sequences facilitating expression of said functional RNA, CP and maturase in a bacterial (e.g. E. coli) expression system. In another embodiment there is provided a host cell comprising the expression vector. In some embodiments, said host cell is a bacterial (e.g. E. coli) cell.

In another aspect there is provided a pharmaceutical composition comprising the nano-carrier system and a pharmaceutically acceptable carrier, excipient or diluent. In various embodiments, the pharmaceutical composition may be used in the treatment of various disorders and conditions, including metabolic disorders (associated with metabolite accumulation and/or aggregation).

In another aspect, the composition is for use in treating a metabolic disorder characterized by elevated metabolite levels in a subject. In another aspect, there is provided a method of treating a metabolic disorder characterized by elevated metabolite levels in a subject, comprising administering to the subject the pharmaceutical composition.

Exemplary disorders and conditions to be treated by the compositions and methods of the invention include, without limitation, adenine phosphoribosyl transferase deficiency (APRT), adenosine deaminase deficiency (ADA deficiency), hypervitaminemia B12, fluoride toxicity, hyperuricemia, gout, Lesch-Nyhan syndrome, glycine encephalopathy, and glutaminase deficiency. Each possibility represents a separate embodiment of the invention.

In one embodiment, the disorder is an inborn error of metabolism (IEM) disorder. In another embodiment, the IEM disorder is selected from the group consisting of APRT and ADA deficiency. In another embodiment, said disorder is APRT, and the riboswitch is an adenine riboswitch. In another embodiment, said disorder is ADA deficiency, and the riboswitch is an adenine riboswitch. In a particular embodiment said disorder is a severe combined immunodeficiency (SCID) resulting from inherited ADA deficiency (ADA-SCID). In another embodiment, said disorder is hypervitaminemia B12 and said riboswitch is a cobalamin riboswitch. In another embodiment, said disorder is fluoride toxicity and said riboswitch is a fluoride riboswitch. In another embodiment, said disorder is hyperuricemia, Lesch-Nyhan syndrome or gout, and said riboswitch is a xanthine I (uric acid) riboswitch. In another embodiment, the disorder is glycine encephalopathy and said riboswitch is a glycine riboswitch. In another embodiment, said disorder is glutaminase deficiency and said riboswitch is glutamine riboswitch.

In another embodiment, the disorder is associated with the formation of metabolite assemblies. In another embodiment said metabolite is a single amino acid or nucleobase. In another embodiment said metabolite is a single amino acid or a nucleobase capable of forming metabolite amyloids in a mammalian cell in vivo. In another embodiment said metabolite is a single amino acid or a nucleobase capable of forming metabolite assemblies that induce apoptotic cell death. In a particular embodiment, the metabolite is adenine. In other embodiments, said metabolite is selected from the group consisting of adenine, tyrosine, and phenylalanine. Each possibility represents a separate embodiment of the invention.

In another aspect, the pharmaceutical composition is for use in treating abnormally elevated metabolite levels in a subject in need thereof. In another aspect, there is provided a method of treating abnormally elevated metabolite levels in a subject in need thereof, comprising administering to the subject the pharmaceutical composition.

In one embodiment, the method or use comprises treating abnormally elevated metabolite levels in the subject's blood or serum. In another embodiment, said VLP is devoid of exogenously added targeting motifs. For example, when the composition is for use in treating abnormally elevated metabolite levels in the subject's blood or serum, the composition to be used may comprise a nano-carrier system of the invention in which the VLP comprises WT MS2 CP is in the form of a single-chain CP dimer optionally comprising a linker between the CP monomer units, wherein said VLP is devoid of exogenously added targeting motifs. In another embodiment said metabolite is B12 and said riboswitch is a cobalamin riboswitch. In another embodiment, said metabolite is uric acid, and said riboswitch is a xanthine riboswitch I.

In another aspect, the pharmaceutical composition is for use in inhibiting or delaying the formation of metabolite assemblies in a subject in need thereof. In another aspect, there is provided a method of inhibiting or delaying the formation of metabolite assemblies in a subject in need thereof, comprising administering to the subject the pharmaceutical composition.

In a particular embodiment of the methods and uses of the invention, the metabolite is adenine, and the riboswitch is an adenine riboswitch.

In another aspect, there is provided a method of treating a metabolic disorder characterized by elevated metabolite levels in a subject, the method comprising administering to the subject a pharmaceutical composition comprising a nano-carrier system, the system comprising: a VLP, and a metabolite-specific functional RNA encapsulated inside the VLP, wherein the functional RNA maintains its ability to selectively bind the metabolite while being encapsulated inside said VLP, and is protected from nuclease degradation in vivo.

Thus, the methods of the invention encompass the use of a metabolite-specific functional RNA capable of selectively binding to the metabolite in question, namely to the metabolite associated with the metabolic disorder to be treated (characteristic by elevated levels of said metabolite). As disclosed herein, the functional RNA maintains its ability to selectively bind the metabolite while being encapsulated inside said VLP, including upon administration to the subject in vivo. Thus, for example, VLPs encapsulating functional RNAs specific to uric acid (e.g. comprising a xanthine riboswitch I capable of binding uric acid with high affinity) may be used in the treatment of conditions associated with elevated uric acid levels such as hyperuricemia, Lesch-Nyhan syndrome and gout, and other disorders as disclosed herein are treated with VLPs comprising functional RNAs specific to the metabolite associated with their etiology or pathology, as detailed hereinbelow.

In another embodiment, said functional RNA comprises a riboswitch and a packing sequence. In another embodiment, said VLP is an MS2 VLP. In another embodiment, said disorder is selected from the group consisting of APRT deficiency, ADA deficiency, tyrosinemia and phenylketonuria. In another embodiment, said subject is afflicted with hyperuricemia, Lesch-Nyhan syndrome and/or gout. In another embodiment, said disorder is selected from the group consisting of hyperuricemia, Lesch-Nyhan syndrome and gout. In another embodiment, said disorder is selected from the group consisting of glutaminase deficiency and glycine encephalopathy. In another embodiment the disorder is associated with the formation of metabolite assemblies. Each possibility represents a separate embodiment of the invention.

In another aspect, there is provided a method of inhibiting or delaying the formation of metabolite assemblies in a subject in need thereof, the method comprising administering to the subject a pharmaceutical composition comprising a nano-carrier system, the system comprising: a VLP, and a metabolite-specific functional RNA encapsulated inside the VLP, wherein the functional RNA maintains its ability to selectively bind the metabolite while being encapsulated inside said VLP, and is protected from nuclease degradation in vivo.

In another embodiment, said functional RNA comprises a riboswitch and a packing sequence. In another embodiment, said VLP is an MS2 VLP. In various embodiments, the metabolite is selected from the group consisting of adenine, tyrosine and phenylalanine. In other embodiments said subject is afflicted with, or is at risk for developing, kidney stones, gallstones, and/or cholesterol deposits. For example, without limitation, kidney stones and gallstones may be associated with the formation of uric acid, adenine and/or oxalate assemblies.

In another aspect, the invention provides a method of reducing the levels of a metabolite in a food product, comprising contacting the product or a precursor thereof with a chelating composition comprising a nano-carrier system, the system comprising:
a VLP, and a metabolite-specific functional RNA encapsulated inside the VLP,
wherein the functional RNA maintains its ability to selectively bind the metabolite while being encapsulated inside said VLP, and is protected from nuclease degradation in vivo.

In one embodiment, said metabolite is selected from the group consisting of lactic acid, ammonia and ionic forms thereof. In another embodiment, said VLP is an MS2 VLP. In another embodiment said food product is a cultured meat product. In another embodiment said product is selected from the group consisting of patties, sausages, burgers, meatballs, cutlets, and hotdogs, wherein each possibility represents a separate embodiment of the invention.

In another aspect, there is provided a nano-carrier system comprising:
a VLP, and a functional RNA encapsulated inside said VLP, wherein said VLP is configured to protect said functional RNA from nuclease degradation in vivo, and wherein said functional RNA maintains its functionality while being encapsulated inside said VLP.

In another embodiment, said VLP comprises a WT MS2 VLP and a packing sequence (bound thereto), and said functional RNA comprises an RNA riboswitch. In a particular embodiment the metabolite is adenine. In various other embodiment, said VLP, functional RNA and metabolite are as disclosed herein.

In another aspect, the invention relates to a method of treating a metabolic disorder in a subject, comprising administering a treatment composition to the subject, wherein the treatment composition comprises a nano-carrier system comprising:
a VLP, and a functional RNA encapsulated inside said VLP, wherein said VLP is configured to protect said functional RNA from nuclease degradation in vivo, and wherein said functional RNA maintains its functionality while being encapsulated inside said VLP.

In another embodiment, said VLP comprises a WT MS2 VLP and a packing sequence, and said functional RNA comprises an RNA riboswitch. In another embodiment the disorder is associated with adenine accumulation and the riboswitch is an adenine riboswitch. In various other embodiment, said VLP, functional RNA and disorder are as disclosed herein.

In another aspect, the invention provides a method of treating an elevated metabolite in a subject, comprising administering a treatment composition to the subject, wherein the treatment composition comprises a nano-carrier system comprising:
a VLP, and a functional RNA encapsulated inside said VLP,
wherein said VLP is configured to protect said functional RNA from nuclease degradation in vivo, and wherein said functional RNA maintains its functionality while being encapsulated inside said VLP.

In another embodiment, said VLP comprises a WT MS2 VLP and a packing sequence and said functional RNA comprises an RNA riboswitch. In various other embodiment, said VLP, functional RNA and metabolite are as disclosed herein.

Other objects, features and advantages of the present invention will become clear from the following description and drawings.

### BRIEF DESCRIPTION OF THE DRAWINGS

FIGS. 1A-1C depict production of VLP-riboswitch nano-carriers within bacteria. FIG. 1A provides a schematic illustration of in vivo VLP-RNA nano-carriers production. FIG. 1B presents a Transmission Electron Microscope (TEM) image of the self-assembled VLPs, following purification from bacterial host (scale bar: 200 nm). FIG. 1C presents dynamic light scattering (DLS) measurements of the VLPs plotted as the relative volume of the VLPs purified (“Volume (%)”) as a function of their diameter (nm).

FIG. 2 presents agarose gel electrophoresis of VLP-RNA nano-carriers (“VLPs”) and naked RNA (“RNA”), respectively, once with RNase A treatment for 1 hr at 37° C. (“RNAse A”) and once without.

FIGS. 3A-3B present TEM images of the Riboswitch-VLP nano-carriers at different post-production time points: FIG. 3A. two weeks post-production (scale-bar: left 100 nm; right 2 μm). FIG. 3B. nine months post-production (scale-bar: left 200 nm; right 1 μm).

FIGS. 4A-4H depict rescue of APT1 and AAH1-deficient yeast by VLP-riboswitch nano-carriers in excess-adenine yeast model. FIGS. 4A-4C present the growth rates (“Growth (OD600)”) of the adenine salvage in vivo aah1Δ apt14 yeast model as a function of time (hr), when grown without adenine and without Riboswitch-VLPs supplementation (“aah1Δ apt1Δ”, solid line), with adenine (“aah1Δ apt1Δ+Ade”, dotted line), and with adenine and Riboswitch-VLPs supplementation (“aah1Δ apt1Δ+Ade+VLPs”, dashed line), respectively. FIG. 4A—1 (mg/L) adenine; FIG. 4B—2 (mg/L) adenine; FIG. 4C—4 (mg/L) adenine. FIGS. 4D-4F are bar diagrams which correspond to FIGS. 4A-4C, respectively, presenting the growth rates (“Growth (OD600)”) of the yeast model (aah1Δ apt1Δ) at particular time points (“hr”) (5, 15, and 25 hours), when grown without adenine and without Riboswitch-VLPs supplementation (“aah1Δ apt1Δ”, solid bar), with adenine (“aah1Δ apt1Δ+Ade”, chequered bar), and with adenine and VLPs (“aah1Δ apt1Δ+Ade+VLPs”, diagonally striped bar), respectively. FIG. 4D—1 (mg/L) adenine; FIG. 4E—2 (mg/L) adenine; FIG. 4F—4 (mg/L) adenine. FIG. 4G presents the percentage of growth at T=25 hours of the mutant yeast with adenine when supplemented with Riboswitch-VLPs (“aah1Δ apt1Δ+Ade+VLPs”, diagonally striped bar) compared to the non-supplemented mutant yeast with adenine (“aah1Δ apt1Δ+Ade”, chequered bar), as a function of adenine concentration. FIG. 4H presents a control experiment showing the growth rates (“Growth (OD600)”) of the adenine salvage in vivo yeast model (aah1Δ apt1Δ) as a function of time (hr), when grown without adenine and without Riboswitch-VLPs supplementation (“aah1Δ apt1Δ”, solid line), and when grown with adenine and VLPs supplementation (“aah1Δ apt1Δ+Ade+VLPs”, dashed line).

FIG. 5 presents the percentage of growth of the mutant yeast with adenine when supplemented with Riboswitch-VLPs (“aah1Δ apt1Δ+VLPs+Ade”, solid grey bar) compared to the non-VLP supplemented mutant yeast with adenine (“aah1Δ apt1Δ+Ade”, solid black bar), at T=25 hours from addition of VLPs, as a function of the confluency of the yeast when the VLPs were added thereto (“Growth (OD600) at VLPs Addition”).

FIGS. 6A-6B depict the effect of VLP-riboswitch nano-carriers on adenine aggregates in aah1Δ apt1Δ yeast model cells. FIG. 6A presents a flow cytometry analysis of aah1Δ apt1Δ cells following ProteoStat staining of amyloid fibrils and aggregates (particularly adenine intracellular aggregates), measuring the FITC (in the X axis) and Cy5-PE (in the Y axis) fluorescence emission of the cells, respectively. Upper left chart: aah1Δ apt1Δ cells without added adenine and without VLP-riboswitch nano-carriers (“−Ade”, “—VLPs”). Upper right: aah1Δ apt1Δ cells with added adenine and without VLPs (“+Ade”, “—VLPs”). Bottom left: aah1Δ apt1Δ cells without adenine and with VLPs (“−Ade”, “+VLPs”). Bottom right: aah1Δ apt1Δ cells with adenine and with VLPs (“+Ade”, “+VLPs”). FIG. 6B presents a comparison between the percent of adenine aggregates in the aah1Δ apt1Δ cells in the presence of adenine (“% of aggregates (proteostat)”), without added VLPs (“−VLPs”, solid bar) and with supplementation of VLP-riboswitch nano-carriers (“+VLPs”, empty bar), as measured in the Flow Cytometry analysis of FIG. 6A (upper right and bottom right, respectively).

FIGS. 7A-7D show the effect of Riboswitch-VLPs as compared to tannic acid in the excess adenine model. FIGS. 7A-7B present the growth rates (“Growth (OD600)”) of the adenine salvage in vivo aah1Δ apt1Δ yeast model as a function of time (hr), when grown without adenine and without VLPs (“aah1Δ apt1Δ”, solid line), with adenine (“aah1Δ apt1Δ+Ade”, dotted line), with adenine and Riboswitch-VLPs supplementation (“aah1Δ apt1Δ+Ade+VLPs”, dashed line), and with adenine and tannic acid supplementation (“aah1Δ apt1Δ+Ade+TA”, solid line with dots), respectively. FIG. 7A—1 (mg/L) adenine; FIG. 7B—4 (mg/L) adenine. FIGS. 7C-7D are bar diagrams corresponding to FIGS. 7A-7B, respectively, presenting the growth rates of the yeast model (aah1Δ apt1) at the endpoint (T=25 hours), when grown without adenine and without VLPs (“aah1Δ apt1Δ”, solid bar), with adenine (“aah1Δ apt1Δ+Ade”, chequered bar), with adenine and Riboswitch-VLPs (“aah1Δ apt1Δ+Ade+VLPs”, diagonally striped bar), and with adenine and tannic acid (“aah1Δ apt1Δ+Ade+TA”, dotted bar), respectively. FIG. 7C—1 (mg/L) adenine; FIG. 7D—4 (mg/L) adenine.

FIGS. 8A-8B depict the effect of VLP-RNA nano-carriers encapsulating a random RNA sequence on the growth of aah1Δ apt1Δ yeast model cells. FIG. 8A presents a TEM image of VLP-RNA nano-carriers encapsulating random RNA sequence containing the TR-RNA activator sequence (scale bar: 100 nm). FIG. 8B presents the growth rates (“Growth (OD600)”) of the adenine salvage in vivo aah1Δ apt1Δ yeast model as a function of time (“Time (hr)”), when grown without adenine and without VLPs supplementation (“aah1Δ apt1Δ”), with adenine (“aah1Δ apt1Δ+Ade”), and with adenine and VLPs encapsulating random RNA (“aah1Δ apt1Δ+Ade+Random VLPs”).

FIGS. 9A-9B present RNA secondary structures of the non-coding RNA functional sequences, calculated by NUPACK platform. FIG. 9A. the TR RNA activator. FIG. 9B. the Adenine Riboswitch A58U.

FIGS. 10A-10G present RNA secondary structures of various RNA constructs including TR-RNA activator and riboswitch sequences, as analyzed by OligoAnalyzer™ Tool or NUPACK platform. FIG. 10A—adenine riboswitch at position 19. FIG. 10B—adenine riboswitch at position 1. FIG. 10C—adenine riboswitch at position 109 (NUPACK). FIG. 10D—40% GC-content in spacer between adenine riboswitch and TR-RNA activator. FIG. 10E—70% GC-content in spacer between adenine riboswitch and TR-RNA. FIG. 10F—secondary structure of cobalamin riboswitch. FIG. 10G—cobalamin riboswitch at position 1.

### DETAILED DESCRIPTION OF THE INVENTION

The invention relates to RNA nanotechnology, in particular to nano-carrier systems comprising a functional RNA encapsulated inside a virus-like particle (VLP), compositions comprising the systems and methods of using same for therapeutic and industrial applications. In particular, the invention in embodiments thereof relates to encapsulation of RNA riboswitches that retain their ability to bind their metabolite ligand and related functionality while being encapsulated inside the VLP, and are protected from nuclease degradation in vivo. Embodiments of the invention further relate to methods of treating and preventing metabolite accumulation in vitro and in vivo, and for the treatment of disorders and conditions associated with metabolite toxicity, accumulation and/or aggregation.

In one aspect, there is provided a nano-carrier system, comprising a functional RNA encapsulated inside an Enterobacteria phage MS2 (MS2) VLP, wherein the functional RNA comprises an RNA riboswitch and a packing sequence, and said riboswitch maintains its functionality while being encapsulated inside said VLP and is protected from nuclease degradation in vivo. In another aspect there is provided a nano-carrier system, comprising a riboswitch encapsulated inside a MS2 VLP. In another embodiment there is provided a nano-carrier system, comprising an adenine riboswitch encapsulated inside a MS2 VLP. In another aspect, there is provided a nucleic acid construct encoding the nano-carrier system. In another embodiment there is provided a host cell comprising the construct.

In another aspect there is provided a pharmaceutical composition comprising the nano-carrier system and a pharmaceutically acceptable carrier, excipient or diluent. In one embodiment the composition comprises a nano-carrier system comprising a functional RNA encapsulated inside an MS2 VLP, wherein the functional RNA comprises an RNA riboswitch and a packing sequence, and said riboswitch maintains its functionality while being encapsulated inside said VLP and is protected from nuclease degradation in vivo. In another embodiment the composition comprises a nano-carrier system comprising a riboswitch encapsulated inside a MS2 VLP. In another embodiment the composition comprises a nano-carrier system comprising an adenine riboswitch encapsulated inside a MS2 VLP.

In another aspect, the pharmaceutical composition is for use in treating a metabolic disorder characterized by elevated metabolite levels in a subject. In another aspect, there is provided a method of treating a metabolic disorder characterized by elevated metabolite levels in a subject, comprising administering to the subject the pharmaceutical composition. In another aspect, the pharmaceutical composition is for use in treating abnormally elevated metabolite levels in a subject in need thereof. In another aspect, there is provided a method of treating abnormally elevated metabolite levels in a subject in need thereof, comprising administering to the subject the pharmaceutical composition. In another aspect, the pharmaceutical composition is for use in inhibiting or delaying the formation of metabolite assemblies in a subject in need thereof. In another aspect, there is provided a method of inhibiting or delaying the formation of metabolite assemblies in a subject in need thereof, comprising administering to the subject the pharmaceutical composition.

In another aspect, there is provided a method of treating a metabolic disorder characterized by elevated metabolite levels in a subject, the method comprising administering to the subject a pharmaceutical composition comprising a nano-carrier system, the system comprising: a VLP, and a metabolite-specific functional RNA encapsulated inside the VLP, wherein the functional RNA maintains its ability to selectively bind the metabolite while being encapsulated inside said VLP, and is protected from nuclease degradation in vivo.

In another aspect, there is provided a method of inhibiting or delaying the formation of metabolite assemblies in a subject in need thereof, the method comprising administering to the subject a pharmaceutical composition comprising a nano-carrier system, the system comprising: a VLP, and a metabolite-specific functional RNA encapsulated inside the VLP, wherein the functional RNA maintains its ability to selectively bind the metabolite while being encapsulated inside said VLP, and is protected from nuclease degradation in vivo.

In another aspect, the invention provides a method of reducing the levels of a metabolite in a food product, comprising contacting the product or a precursor thereof with a chelating composition comprising a nano-carrier system, the system comprising: a VLP, and a metabolite-specific functional RNA encapsulated inside the VLP, wherein the functional RNA maintains its ability to selectively bind the metabolite while being encapsulated inside said VLP, and is protected from nuclease degradation in vivo.

In another aspect, there is provided a nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP.

In another aspect, the invention relates to a method of treating a metabolic disorder in a subject, comprising administering a treatment composition to the subject, wherein the treatment composition comprises a nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP. In another aspect, the invention provides a method of treating an elevated metabolite in a subject, comprising administering a treatment composition to the subject, wherein the treatment composition comprises a nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP.

These and other embodiments are described in greater detail and exemplified below.

### Functional RNA

According to embodiments of the invention, the invention relates to a functional RNA molecule that may be encapsulated in a VLP. In some embodiments, the functional RNA is, or comprises, a riboswitch. In other embodiments, the functional RNA comprises at least the ligand-binding domain of a riboswitch, or in other embodiments, a synthetic analog or derivative of the riboswitch or its ligand-binding domain. Each possibility represents a separate embodiment of the invention. Metabolite-sensing RNAs, or “riboswitches,” are structured noncoding RNA molecules that specifically interact with small-molecule or ion cellular metabolites, and are capable of directing or regulating expression of the genes involved in their metabolism. The structure of a riboswitch includes the evolutionarily conserved ligand-binding domain (also known as the aptamer domain) and the expression platform, both of which are connected by the switching sequence. Conserved features of the ligand-binding domain include nucleotide sequences (particularly at or near the ligand-binding core), secondary structures such as base-paired stems and pseudoknots, and motifs such as k-turns, E-loops, special tetraloops, and other common substructures. Binding of a metabolite induces a structural (allosteric) change in the expression platform, which is a typically a variable region.

The two distinct functional states of riboswitches depend on metabolite binding, with the notable exception being the adenine riboswitch present in the add gene of the thermophile Vibrio vulmficus, in which the occurrence of three stable conformations was demonstrated, allowing environmental-dependent ligand recognition. Thus, riboswitches are characterized in that a single RNA sequence is capable of adopting, at least, two stable secondary structures in order to regulate the expression of a given gene. These structures are conserved throughout evolution despite sequence variations.

Riboswitches are typically classified according to the type or identity of their effector ligand; being evolutionarily conserved, riboswitches directed to a common ligand are also characterized by shared structural features. Thus, riboswitches are a group of noncoding RNA molecules characterized by unique structural and functional properties. Riboswitch structures and mechanisms are described in Garst et al, 2011 ibid, and the consensus sequence and secondary structures characteristic of the predominant riboswitch classes and their respective domains are described, for example, in McCown et al., 2017 (RNA 23: 995-1011), both incorporated herein by reference.

The largest known class includes riboswitches capable of recognizing coenzymes, including, but not limited to, Thiamin pyrophosphate (TPP) riboswitch and HMP-PP riboswitch (bind TPP); AdoCbl and AqCbl Cobalamin riboswitches (bind Coenzyme B12); S-Adenosylmethionine (SAM) riboswitch I-VI, SAM-SAH riboswitch and SAH riboswitch (bind SAM); Flavin mononucleotide riboswitch (binds FMN); Tetrahydrofolate (THF) riboswitch I and II (bind THF); Molybdenum cofactor (MoCo) riboswitch; Tungsten cofactor (WCo) riboswitch; and Nicotinamide adenine dinucleotide (NAD⁺) riboswitch I and II (bind NAD⁺).

Riboswitches belonging to the second largest group bind purines and some derivate purine compounds including, but not limited to adenine, guanine, pre-queuosine-1 (preQ1), deoxyguanosine (dG), cyclic-di-GMP (c-di-GMP), and cyclic-di-AMP. Riboswitches belonging to classes that bind nucleotides and precursors and derivatives thereof, include, but are not limited to, Guanine riboswitch, Adenine riboswitch, Prequeuosine-1 (preQ₁) riboswitches I-III (bind preQ₁), 2′-Deoxyguanosine (2′-dG) riboswitch, ZTP riboswitch, Phosphoribosyl pyrophosphate (PRPP) riboswitch, Phosphoribosylamine (PRA) riboswitch, Adenosine 5′-diphosphate (ADP) riboswitch, Xanthine riboswitch I (binds xanthine, uric acid, and 8-azaxanthine), and Xanthine riboswitch II. Most of these riboswitch ligands could alternatively be categorized as including purines, and their biosynthetic intermediates. The sequence of the ligand-binding domain of the purine riboswitch contains three conserved helical elements (P1, P2, and P3). These form sets of coaxial stacks in which two or more individual helices are arranged in a single pseudocontinuous helix, exemplified by the P1 and P3 stack.

Other riboswitch classes recognize amino acids, including lysine, glycine, and glutamine; riboswitch classes which bind amino acids, include but are not limited to, Glutamine riboswitch I and II (bind glutamine), Lysine riboswitch, and Glycine riboswitch. Other riboswitch classes bind ions, including, but not limited to Mg²⁺ riboswitch I and II (bind Mg²⁺), Mn²⁺ riboswitch, Fluoride riboswitch (binds F⁻), NiCo riboswitch (binds Ni²⁺/Co²⁺), Li⁺ riboswitch I and II (bind Li⁺), and Na⁺ riboswitch I and II (bind Na⁺). Additional riboswitch classes bind common nucleotide-based signaling molecules, including, but not limited to Cyclic di-GMP (c-di-GMP) riboswitch I and II (bind c-di-GMP); Cyclic di-AMP (c-di-AMP) riboswitch; Cyclic AMP-GMP (c-AMP-GMp) riboswitch; Guanosine tetraphosphate (ppGpp) riboswitch; and ZTP riboswitch.

In some embodiments, the riboswitch used in the systems and methods of the invention is a naturally-occurring riboswitch, e.g. of a bacterial or archaeal source. In other embodiments, said riboswitch is a synthetic riboswitch, derived from a sequence of a naturally occurring riboswitch and including e.g. one or more point mutations (e.g. enhancing its stability or ligand affinity), while maintaining the conserved sequence motifs and secondary structures of the naturally-occurring riboswitch. In certain particular embodiments, the ligand-binding domain of the synthetic riboswitch has a sequence of at least 90% (e.g. 93%, 95%, 97%, 98%, or 99%) identity to the ligand-binding domain of the corresponding naturally-occurring riboswitch.

In some embodiments of the present invention, the riboswitch to be used in the systems and methods of the invention is an adenine riboswitch. The adenine riboswitch selectively recognizes adenine (due to the “UUU” motif including a uracil ribonucleotide in position 74 of the adenine-binding pocket), and is often found, in its naturally occurring state, in the 5′ UTR of bacterial mRNAs of genes which regulate adenine levels. The secondary structure of the adenine riboswitch includes a three-stem junction, wherein two of the three stems extending from the junction each form a “stem-and-loop” conformation, referred to herein also as “arms” of the riboswitch, and the three joining regions largely form the adenine-binding pocket of the riboswitch (Garst et al, 2011 ibid; McCown et al., 2017 ibid). Two well studied adenine riboswitches include the add adenine riboswitch, naturally occurring in the UTR of the gene which translates into adenosine deaminase (add) enzyme (e.g. from. Vibrio vulmficus), and the pbuE adenine riboswitch, naturally occurring in the UTR of the gene which encodes a purine base efflux pump (pbuE, e.g. from B. subtilis). The add adenine riboswitch has three stable conformations, two in an unbound state, i.e., when not binding adenine, and the third conformation when binding adenine. In one of the two unbound conformations the add adenine riboswitch is not able to accept adenine into its binding pocket. The affinity of add adenine riboswitch to adenine is also influenced by temperature and magnesium concentration, generally expressing an enhanced affinity at temperatures of between 30° C.-40° C. and at high Mg²⁺ concentrations. In contradistinction, the pbuE adenine riboswitch has only two stable conformations, alternating between them depending on whether it is binding adenine or not, which are the common conformation types in most identified riboswitches to date.

In some embodiments, the riboswitch is a naturally occurring adenine riboswitch (e.g. an add or pubE riboswitch as disclosed herein). In another embodiment said riboswitch is a synthetic adenine riboswitch. For example, the pbuE adenine riboswitch has been studied for the effect of point mutations on the affinity for adenine, specifically in the context of the dissociation constant K_{D}. A point mutation replacing the Adenine nucleobase (A) at position 58 of the riboswitch with a Uracil nucleobase (U) (this mutation is referred to herein as “A58U riboswitch”, “A58U adenine riboswitch”, or just “A58U”), has been found to reduce the dissociation constant from K_{d}=354±17 nM, of the wild type pbuE adenine riboswitch, to K_{d}=273±17 nM, i.e., increases the affinity of the riboswitch to its adenine ligand. Additional point mutations in the pbuE riboswitch have been found which increase or decrease the dissociation constant of the pbuE adenine riboswitch to adenine. The term “adenine riboswitch” as used throughout the specification may refer to any one or more of the types of naturally occurring or synthetic adenine riboswitch, unless otherwise stated or otherwise understood by the context.

In some embodiments of the present invention, the riboswitch used is Cobalamin riboswitch. The cobalamin riboswitch selectively recognizes cobalamin (known also as “vitamin B12”, or “coenzyme B12”), and is widely distributed, in its naturally occurring state, in the 5′ UTR of cobalamin related genes in bacteria, particularly genes related to cobalamin metabolism.

Cobalamin is a complex enzyme cofactor, which appears in several different forms, including (but not limited to): adenosylcobalamin (AdoCbl), methylcobalamin (MeCbl), hydroxocobalamin (HyCbl), and cyanocobalamin (CyCbl). The main class of cobalamin riboswitch, termed AdoCbl riboswitch, is divided into several sub-classes according to the respective cobalamin analogue selectivity, as well as differences in peripheral structural elements. The sub-classes include class-I cobalamin riboswitch (Cbi-I), class-II cobalamin riboswitch (Cbi-II), and Cbi-II riboswitch is further includes Cbi-IIa riboswitch and Cbi-IIb riboswitch. Aquocobalamin is another form of cobalamin (AqCbl), which is bound by a distinct riboswitch class referred to as AqCbl riboswitch. The term “cobalamin riboswitch” as used throughout the specification refers to any one of the above-mentioned classes or sub-classes of the cobalamin riboswitch, unless expressly stated otherwise.

In another embodiment, the riboswitch used is a Xanthine riboswitch. Xanthine riboswitch I includes a highly conserved NMT1 RNA motif, has been identified in at least 650 proteobacteria strains, and selectively binds xanthine (a guanine derivative, (KD) ˜3.7 μM), uric acid (a derivative of xanthine, (KD) ˜25 μM), and 8-azaxanthine (a small molecule inhibitor of uric acid oxidase). The secondary structure of the xanthine riboswitch I forms a three-stem junction, wherein the loops of the two “arms” form a pseudoknot (kissing-loop structure) and the three joining regions largely form the xanthine-binding pocket of the riboswitch.

In another exemplary embodiment, the riboswitch used is a fluoride riboswitch. The fluoride riboswitch is a conserved RNA structure identified in a wide variety of bacteria and archaea, which binds fluoride ions. The structure of the fluoride riboswitch is recognized to include two helical stems adjoined by a helical loop, with the capacity to become a pseudoknot. The ability of the riboswitch to bind the negative fluoride ion, despite the negative charge of the RNA phosphate groups themselves, is facilitated by three Mg2⁺ ions residing in the fluoride-binding pocket of the riboswitch. In various other embodiments, said riboswitch is a glycine riboswitch, a FMN riboswitch or a Glutamine riboswitch (Garst et al, 2011 ibid; McCown et al., 2017 ibid). In another embodiment, said riboswitch belongs to a class as disclosed herein. In other embodiments, said riboswitch to be used in the systems and methods of the invention is a riboswitch as disclosed herein. Each possibility represents a separate embodiment of the invention.

In other embodiments, the functional RNA further comprises a packing sequence (also referred to as a capsid packing sequence or a viral packing signal). More specifically, the region(s) of viral RNA responsible for directing packaging is referred to as either the packaging sequence or signal or the encapsidation sequence. Spherical, positive-sense, single-stranded RNA (ssRNA) viruses, which include many major pathogens in all kingdoms of life, assemble by the co-assembly of coat protein (CP) subunits around the genome. Most families of these viruses encode single-copy, high affinity CP-binding sites in their genomes that act as assembly initiation sites. These RNA sequences form defined secondary structure elements that are specifically recognized by the viral CPs. The best characterized of these interactions is the 19-nt-long stem-loop named TR in the bacteriophage MS2 genome. Thus, in more specific embodiments, the packaging sequence comprised within the functional RNA may be at least one TR-RNA activator sequence (also referred to as “TR-RNA”). In some embodiments, the packing sequence used in the systems and methods of the invention may be a bacteriophage packaging signal of at least one bacteriophage of the Leviviridae family, e.g. MS2 TR-RNA. In a particular embodiment said packaging sequence is a WT MS2 TR-RNA. In other embodiments other TR-RNA sequences that are used as packaging signals in connection with the particular VLP CP employed, may be applicable in the present invention, that is not limited to the TR-RNA disclosed and exemplified by the invention. It is to be understood, however, that the TR-RNA to be used in the systems and methods of the invention is selected such that it specifically binds the VLP. In some embodiments the TR-RNA may comprise the nucleic acid sequence as denoted by at least one of SEQ ID NO: 2, or any variants and analogs thereof (e.g. having at least 90%, 93%, 95%, 97% or 99% sequence identity, and which retains the secondary structure and target specificity of the native TR-RNA). Each possibility represents a separate embodiment of the invention.

Accordingly, disclosed herein is the use of recombinant, non-naturally occurring non-coding RNA molecules, that possess functional properties as disclosed herein under physiological conditions, termed functional RNA or functional RNA constructs. In particular, a functional RNA to be used in the systems and methods of the invention is a recombinant single-stranded noncoding RNA molecule comprising a plurality of structurally and functionally distinct elements. These elements typically include at least a metabolite-binding element (having metabolite-binding activity) and a packaging sequence (having a capsid-binding activity facilitating encapsulation). In particular, advantageous functional RNA molecules comprise a metabolite-binding element in the form of a riboswitch as disclosed herein and a packing sequence such as a TR-RNA.

Further, as disclosed herein, functional RNA molecules of the invention maintain the aforementioned functions while being encapsulated in a VLP in accordance with the invention (including under physiological conditions in vivo). In the case of a riboswitch, the function typically further include a conformational (allosteric) change induced by the metabolite ligand binding, as discussed above. It is to be understood, however, that while the expression-modulation properties of a riboswitch (facilitated by ligand binding) may be preserved in the encapsulated functional RNA, they are not typically required for the intended activity of the systems and methods of the invention. In some embodiments, the systems of the invention are used in methods by which the functional RNA does not bind directly to a gene or transcript thereof in the target cell, and does not directly affect transcription, translation or splicing of said gene or transcript. In some embodiments, the systems and methods of the invention employ the use of functional RNA molecules maintaining the VLP and ligand binding properties, and are substantially devoid of additional activities at their intended target (e.g. the patient to be treated), such as enzymatic activity and direct expression-modulation activity.

Thus, a functional RNA maintaining its functionality while being encapsulated inside said VLP meaning that the aforementioned functions are qualitatively preserved. In addition, the term indicates that these functions and in particular the metabolite-binding property are substantially quantitatively preserved. Thus, for example, the metabolite-binding activity of the riboswitch may be at least 60%, 70%, 80%, 90%, 95% or 98% of that of the non-encapsulated riboswitch under nuclease-free conditions. In other embodiments, the ligand-binding activity of the functional RNA while being encapsulated inside the VLP is about 20%, 30%, 40% 50% higher than that of the non-encapsulated RNA, or, in other embodiments, twofold, threefold, fourfold or tenfold higher. Remarkably, as disclosed herein, despite comprising multiple secondary and tertiary structures and despite extensive allosteric structural alterations required for their activity, the invention in embodiments thereof discloses the construction and use of riboswitch-containing functional RNAs, in which the individual functional properties associated with the distinct structures are maintained.

Typically, functional RNA of the invention is characterized in that the riboswitch (or other metabolite-binding element as disclosed herein) is located 5′ to the TR-RNA. In one embodiment, said riboswitch (or element) and TR-RNA are separated by a non-coding RNA spacer. Typically, the RNA spacer is longer than about 50 nucleotides (nt) in length, more typically longer than 80, 100 or 120 nt in length. In some embodiments, said spacer is up to about 300 nt in length, or in other embodiments up to about 250 or 200 nt in length. In another embodiment said RNA spacer is characterized by a length of 100-200 nt in length, e.g. 100-170, 120-170, 120-200, or 130-160 nt in length. In another embodiment said spacer is characterized by a guanine-cytosine content (GC content) of 20%-70%, e.g. 30%-70%, 20-65%, 30%-65%, 40-65%, or 40%-60%. In another embodiment, said riboswitch is located less than 50, 40, 30 or 20 nt from the 5′ end. In some embodiments, the region 5′ to the riboswitch or metabolite-binding domain is a noncoding RNA sequence characterized by a GC content of 20%-70%, e.g. 30%-70%, 20-65%, 30%-65%, 40-65%, or 40%-60%. Each possibility represents a separate embodiment of the invention. The term “GC content” refers to the percentage of a nucleic acid sequence comprised of deoxyguanosine (G) and/or deoxycytidine (C) deoxyribonucleosides, or guanosine (G) and/or cytidine (C) ribonucleoside residues, depending on the context (e.g. when describing DNA or RNA).

In an exemplary embodiment, the riboswitch is located 5′ to the TR-RNA, and said riboswitch and TR-RNA are separated by a non-coding RNA spacer characterized by a length of 100-200 nt and a GC content of 20%-70%. In another exemplary embodiment, said riboswitch is located less than 30 nt from the 5′ end, said TR-RNA is located at the 3′ end, and the spacer is 120-170 nt in length and has a GC content of 40%-65%. In another embodiment, said functional RNA has a nucleic acid sequence selected from the group consisting of SEQ ID NOs: 6, 7, 9-13, 19-21 and 23, as set forth and exemplified in Example 5.

### VLPs

As disclosed herein, nano-carrier systems of the invention comprise a functional RNA encapsulated inside a VLP. The term “encapsidate” or “encapsulate” refers to enclosure of a nucleic acid molecule within a structure comprising the virion structural proteins of a virus or VLP (i.e. the capsid of said virus or VLP). As used herein, the term VLP denotes a nanoparticle comprising the aforementioned capsid and that does not contain the native viral genome.

In one embodiment, the VLP is an Enterobacteria phage MS2 (MS2) VLP. In another embodiment, the VLP capsid is derived from another bacteriophage of the Leviviridae family. Bacteria, including Enterobacteria, Caulobacter, Pseudomonas, and Acinetobacter serve as natural hosts for Leviviridae bacteriophages. There are currently four species in this family, divided among two genera. They are small RNA viruses with linear, positive-sense, single-stranded RNA genomes that encode only four proteins. All phages of this family require bacterial pili to attach to and infect cells. In some embodiments, the bacteriophage of the Leviviridae family may be a bacteriophage of the genus Leviviru. Enterobacteria serve as natural hosts of Leviviru bacteriophages. There are currently only two species in this genus including the type species bacteriophage MS2. The bacteriophage MS2 is an icosahedral, positive-sense single-stranded RNA virus that infects the bacterium Escherichia coli and other members of the Enterobacteriaceae. MS2 is a member of a family of closely related bacterial viruses that includes bacteriophage f2, bacteriophage Q, #, R17, and GA.

An MS2 virion (viral particle) is about 27 nm in diameter, as determined by electron microscopy. It consists of one copy of the maturation protein and 180 copies of the coat protein (CP; organized as 90 dimers) arranged into an icosahedral shell with triangulation number T=3, protecting the genomic RNA inside. The virion has an isoelectric point (Pi) of 3.9. The structure of the coat protein is a five-stranded β-sheet with two α-helices and a hairpin. When the capsid is assembled, the helices and hairpin face the exterior of the particle, while the β-sheet faces the interior.

In another embodiment, said VLP comprises a capsid comprising wild-type (WT) MS2 coat proteins (CP). In one embodiment, the MS2 VLP is a WT MS2 VLP. As used herein, a WT MS2 VLP is a virus-lite particle in which at least the CP corresponds to a CP monomer of a wild-type MS2 virus. In another embodiment, said VLP consists essentially of WT MS2 CP units (monomers). In another embodiment said WT MS2 CP is in the form of a single-chain CP dimer, which may comprise a linker between the CP monomer units. In another embodiment, said CP or CP dimer further comprises at least one peptide tag (e.g. a histidine tag or an HA tag). An amino acid sequence of the WT MS2 CP monomer is set forth in SEQ ID NO: 17, as follows:

(SEQ ID NO: 17)
MASNFTQFVLVDNGGTGDVTVAPSNFANGVAEWISSNSRSQAYKVTCSVR
QSSAQNRKYTIKVEVPKVATQTVGGVQLPVAAWRSYLNMELTIPIFATND
DCALIVKAMQGLLKDGNPIPSAIAANSGIY.

As disclosed herein a VLP comprised of CP dimer subunits derived from WT MS2 was found to be configured to protect a functional RNA comprising an adenine riboswitch and an MS2 TR-RNA from nuclease degradation in vivo (as determined in a yeast model system). Thus, VLPs in accordance of the invention advantageously comprise a WT MS2 CP, or in other embodiments, homologous CP that substantially maintain the structural and functional properties of the MS2 VLP as disclosed herein. According to particular embodiments, the CP may have at least 87%, 90%, 93%, 95%, 97% or 99% sequence identity to a WT MS2 CP monomer (SEQ ID NO: 17).

In yet other embodiments, the VLP is comprised of a CP dimer encoded by a sequence as set forth in SEQ ID NO: 4, or having at least 87%, 90%, 93%, 95%, 97% or 99% sequence identity thereto.

Still further, in some embodiments, the linker linking between the at least two MS2 CPs, or alternatively, the CP-dimer itself may comprise or alternatively, directly or indirectly connected to at least one tagging moiety and/or at least one targeting moiety. Thus, in certain embodiments, the linker or CP-dimer, may further comprise at least one of, at least one tagging moiety and at least one targeting moiety. In some embodiments, the linker may be tagged. Different tags or tagging moieties may be also used, for example, His, myc, HA, GFP, ABP, GST, biotin, FLAG and the like.

Still further, in some embodiments, the CP-dimers or the connecting linker/s may further comprise at least one directly or indirectly attached targeting moiety. This enables creation of VLPs composed of the labeled CP-dimers and comprising the encapsulated functional RNA, that are targeted to a specific target in a desired cell, tissue or organ. The term “targeting moiety” (also referred to herein as an exogenous or heterologous targeting moiety) relates to an affinity molecule that can target the VLPs to a specific target e.g. a tissue. In certain embodiments, the targeting moiety may be an antibody or an aptamer.

The terms “antibody” or “antibodies” as used herein refer to an antibody, preferably a monoclonal antibody, or fragments thereof, including, but not limited to, a full-length antibody having a human immunoglobulin constant region, a monoclonal IgG, a single chain antibody, a humanized monoclonal antibody, an F(ab′)₂ fragment, an F(ab) fragment, an Fv fragment, a labeled antibody, an immobilized antibody and an antibody conjugated with a heterologous compound. Each possibility represents a separate embodiment of the invention. In one embodiment, the antibody is a monoclonal antibody (mAb). In another embodiment, the antibody is a polyclonal antibody. In another embodiment, the antibody is a humanized antibody. Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1).

In yet some further embodiments, as targeting moiety, the CP-dimers encoded by the nucleic acid sequences of the systems of the invention may comprise at least one aptamer. As used herein the term “aptamer” or “specific aptamers” denotes single-stranded nucleic acid (DNA or RNA) molecules which specifically recognizes and binds to a target molecule. The aptamers may fold into a defined tertiary structure and can bind a specific target molecule with high specificities and affinities. Aptamers are usually obtained by selection from a large random sequence library, using methods well known in the art, such as SELEX and/or Molinex.

Yet in other advantageous embodiments, the CP, CP-dimers and VLP are devoid of any exogenously added (heterologous) targeting moiety, not naturally occurring in the corresponding WT CP. Thus, as disclosed herein, systems in accordance with the invention may be used without the need of being internalized into a particular cellular target, and accordingly the functional RNA encapsulated in the VLP may advantageously exert its functional activity without being introduced or endocytosed to the cell, and while remaining encapsulated inside the VLP.

In another embodiment, the VLP further comprises a packing sequence (that is typically bound to a CP component thereof). In certain embodiments, the VLP may further comprise an additional viral protein. In some embodiments, such additional viral protein may be the MS2 maturase. MS2 Maturase or, Maturation protein A is an 44.9 Kd protein required for the typical attachment of the phage to the side of the bacterial pili, and is considered to be involved in cell entry (PubMed:26608810).

As is further demonstrated herein, the encapsulated functional RNA including in particular the riboswitch element thereof is protected from nuclease degradation in vivo. Thus, said RNA or riboswitch remain substantially intact and active under physiological conditions characterized by high nuclease (including endonuclease) concentrations characteristic of cells, tissues and blood circulation of living organisms for a time duration sufficient to exert a therapeutic or otherwise physiologically significant effect (e.g. at least 1, 2, 4, or 24 hours). As demonstrated herein, the nano-carrier systems of the invention remained active when applied in an in vivo yeast culture model for over 24 hours. As further demonstrated, this effect was correlated with the ability of the nano-carrier system to substantially retain RNA integrity for at least one hour at 37° C. in vitro, in the presence of 10 U of RNase A (i.e., a concentration considerably higher than the physiological nuclease concentration in vivo, and sufficient to completely degrade non-encapsulated RNA under the same conditions). Thus, protection from nuclease degradation in vivo may conveniently be evaluated by an in vitro assay as specified above, in which at least 50% and typically at least 60%, 70%, 80%, 90% or 95% of said RNA remains non-degraded. Methods and means for performing such assays are exemplified in the Examples below.

### Nucleic Acid Constructs and Recombinant Methods

In other embodiments, the invention relates to a nucleic acid construct encoding the nano-carrier system as disclosed herein. In some embodiments, the nucleic acid construct may be used for production and assembly of the nano-carrier system using various synthetic and/or recombinant production methods and expression systems.

Polypeptides and peptides may conveniently be produced by recombinant technology. Recombinant methods for designing, expressing and purifying proteins and peptides are known in the art (see, e.g. Sambrook et al., 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, modified nucleic acid sequences in which nucleotides have been inserted, deleted, substituted, and/or inverted in such a manner that such modifications do not substantially interfere with the nucleic acid molecule's ability to encode a functional product. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide and the polypeptide is synthesized in the host upon transcription and translation of the DNA sequence.

In some embodiments, the constructs of the invention comprise at least one nucleic acid sequence encoding the functional RNA, the capsid proteins (e.g. CP) and optionally the maturase, which are conveniently operably linked to one or more transcription regulating sequences. The term “operably linked” refers to a functional linkage of at least two sequences. Operably linked includes a linkage between a promoter and a second sequence, for example a nucleic acid of the present invention, wherein the promoter sequence initiates and mediates transcription of the DNA sequence corresponding to the second sequence. In various embodiments, the transcription regulating sequence may include inducible or constitutive promotors, enhancers, repressors, or post translationally regulatory elements such as for example degrons. As used herein, a “promoter sequence” is a DNA regulatory region capable of binding RNA polymerase in a cell and initiating transcription of a downstream (3′ direction) coding sequence. A “constitutive promoter” refers to a promoter that allows for continual transcription of the coding sequence or gene under its control. Exemplary suitable constitutive promoters include e.g. E. coli promoters such as P(Bla), P(Cat) and P(Kat). An “inducible promoter” refers to a promoter that initiates increased levels of transcription of the coding sequence or gene under its control in response to a stimulus or an exogenous environmental condition. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the T7 polymerase promoter system (WO 98/10088), the tetracycline-repressible system, the tetracycline-inducible system and the lac promoter. Additional inducible promoters (e.g. for mammalian expression systems) include e.g. the RU486- and rapamycin-inducible systems.

A construct of the invention intended for production in a prokaryotic expression system may contain a bacterial promoter, such as a lac promoter or it may contain a bacteriophage promoter, such as a T7 promoter and optionally a T7 transcription terminator. In some embodiments, advantageous promoters include, for instance, T7, T3, and SP6 promoters.

VLPs may be synthesized in vitro in a coupled cell-free transcription/translation system. Alternatively, VLPs could conveniently be produced in vivo by introducing transcription units into bacteria, especially if transcription units contain a bacterial promoter. For example, capsids and RNA molecules may be co-expressed in any expression system. Recombinant DNA encoding one or more capsid proteins, and the functional RNA can be readily introduced into the host cells, e.g., bacterial cells, plant cells, yeast cells, fungal cells, and animal cells (including insect and mammalian cells) by transfection with one or more expression vectors by any procedure useful for introducing such a vector into a particular cell, and stably transfecting the cell to yield a cell which expresses the recombinant sequence(s).

For in vitro manufacture, purified coat protein subunits may be obtained from VLPs that have been disaggregated with a denaturant (usually acetic acid), and subsequently mixed with the functional RNA. VLPs may also be synthesized in a coupled in vitro transcription/translation system using procedures known in the art. In a particular embodiment, bacteriophage T7 (or a related) RNA polymerase is used to direct the high-level transcription of genes cloned under control of a T7 promoter in systems optimized to efficiently translate the large amounts of RNA thus produced. In some embodiments, in which the VLP is formed in a bacterial expression system, expression of the capsid protein and the RNA sequence may be induced by the addition of a suitable amount of inducer (e.g. Isopropyl β-D-1-thiogalactopyranoside (IPTG)) under conditions sufficient to produce large quantities of the desired capsid protein and RNA. The capsid protein and RNA spontaneously form VLPs within the E. coli host cell, and after a suitable period the host cells comprising the assembled VLPs are isolated and processed.

Following the production step, the resulting VLPs encapsulating the functional RNA are typically isolated, recovered and/or purified, to be used in the methods of the invention. In some embodiments, the resulting VLPs may be isolated using affinity column such as Nickel beads (Ni) column. Purification of capsids, VLPs or proteins may also include methods generally known in the art. For example, following capsid expression and purification of capsids, VLPs or proteins may include for example at least one liquid-liquid extraction step, at least one fractional precipitation step, at least one ultrafiltration step, at least one ultracentrifugation step, at least one gradient elution step and/or at least one crystallization step. Liquid-liquid extraction may comprise e.g. use of an immiscible non-aqueous non-polar solvent, e.g. benzene, toluene, hexane, heptane, octane, chloroform, dichloromethane, or carbon tetrachloride. Non-limiting examples for VLP production in an E. coli system and subsequent isolation are provided in the Examples below.

### Pharmaceutical Compositions

In in some embodiments, the invention relates to a pharmaceutical composition comprising a nano-carrier system of the invention and a pharmaceutically acceptable carrier, excipient or diluent. In a particular embodiment, the pharmaceutical composition is formulated for administration by injection, e.g. for intravenous administration. In other embodiments, said pharmaceutical composition is formulated for oral administration.

Pharmaceutical compositions disclosed by the invention used to treat subjects in need thereof according to the invention may comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

“Pharmaceutically or therapeutically acceptable carrier” refers to a carrier medium which does not interfere with the effectiveness of the biological activity of the active ingredients. As mentioned herein, the compositions provided by the invention optionally further comprise at least one pharmaceutically acceptable excipient or carrier. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated. As used herein “pharmaceutically acceptable carrier/diluents/excipient” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

Oral compositions will generally include an inert diluent or an edible carrier and may be compressed into tablets or enclosed in gelatin capsules. Tablets, pills, capsules, troches and the like can contain any of the following ingredients, or compounds of a similar nature: a binder, such as microcrystalline cellulose, gum tragacanth and gelatin; an excipient such as starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring.

Further, the pharmaceutical composition (also referred to as a “treatment composition”) contains the nano-carrier system at a therapeutically effective amount. The terms “effective amount” or “therapeutically effective amount” mean an amount necessary to achieve a selected result. In the context of the present invention, the amount is selected to be effective in treating a condition or disorder as disclosed herein. The effective amount is determined by the severity of the disease in conjunction with the preventive or therapeutic objectives, the route of administration and the patient's general condition (age, sex, weight and other considerations known to the attending physician).

### Therapeutic Use

In various embodiments, the systems and methods of the invention are used in the management of disorders, conditions and complications associated with the excessive or abnormal presence of metabolites within the body of a subject, or in a particular tissue and/or bodily fluid (e.g. blood serum).

As used herein, the term metabolite encompasses small molecule substances used in or produced from cellular metabolism. The term metabolite is generally restricted to organic and inorganic small molecules and does not include polymeric compounds such as DNA and proteins. A metabolite may serve as a substrate for an enzyme of a metabolic pathway, an intermediate of such a pathway or the product obtained by the metabolic pathway. In some embodiments, the term metabolite refers in particular to non-proteinaceous organic small molecules (including, but not limited to nucleobases, amino acids and derivatives thereof) capable of self-assembly to form metabolite aggregates in vivo, as will be discussed in greater detail below. In a particular embodiment, the metabolite is a riboswitch ligand as disclosed herein.

Certain metabolites may be a direct and/or indirect cause of a health disorder in a subject, when present in excess. For example, the excess molecules may be precursors of other molecules which cause a damaging or pathological effect to the subject, and/or they may themselves produce the damage, pain or discomfort. For the sake of clarity, the term “metabolites” or “elevated metabolites” as will be further referred to below relate to those metabolites that may contribute to the etiology or pathology of a disorder or adverse health condition in a subject. Exemplary metabolites include, but are not limited to, adenine, glycine, glutamine, phenylalanine, tyrosine, uric acid, vitamin B12, and fluoride (F⁻ ion).

A condition of elevated metabolites may be the result of a variety of different causes, such as (but not limited to) behavioral habits of the afflicted subject (e.g. nutritional habits or exercise), malfunction of body organs (e.g., kidneys), and/or genetic defects.

In some embodiments, the systems and methods of the invention are used in the treatment of a metabolic disorder in a subject. As used herein, the term metabolic disorder refers to a pathological condition associated with metabolite accumulation and/or aggregation. In other words, metabolic disorders to be treated by the systems and methods of the invention involve diseases and conditions in which elevated metabolite levels and/or the formation of metabolite assemblies contribute to the development of the disorder, as well as diseases and conditions in which such abnormally elevated metabolite levels are manifested among the symptoms and signs of said disorder. Typically, the metabolic disorder is a result of inability of the body to perform normal biochemical reactions related to one or more biosynthetic pathways associated with the metabolite.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

In certain embodiments, the systems and methods of the invention are used in the treatment of a metabolic disorder characterized by elevated metabolite levels in a subject (as a pathophysiological manifestation). Metabolic disorders which are due to inborn genetic defects which impair normal enzymatic activity are sometimes referred to herein as “inborn errors of metabolism” (IEM). Error of metabolism disorders arise from an inability of the cellular machinery to perform critical biochemical reactions along a biosynthetic pathway. In the case of IEM disorders, most disorders are due to a genetic mutation leading to the malfunction of an enzyme crucial for a metabolic process. This, in turn, results in metabolite accumulation and tissue damage that can lead to mental retardation, epilepsy, and organ damage. For example, IEM disorders associated with accumulation of adenine or derivatives thereof, include adenosine deaminase (ADA) deficiency and adenine phosphoribosyltransferase (APRT) deficiency.

In some embodiments, the systems and methods of the invention are utilized for the treatment of APRT. APRT is an autosomal recessive IEM which affects the kidneys and urinary tract. The cause of APRT is a mutation in the APRT gene, which hampers the production of a functional APRT enzyme. A lack of functional enzyme impairs the conversion of adenine to AMP, catalyzed by the APRT enzyme. As a result of the accumulation of non-converted adenine, it is converted instead into 2,8-dihydroxyadenine (2,8-DHA), which crystallizes in urine, forming stones in the kidneys and urinary tract. Common manifestations of APRT deficiency include kidney and urinary tract stones, and may further result in urinary tract infection, fever, blood in the urine (hematuria), abdominal cramps, nausea, and vomiting. At present, the commonly used treatment for APRT involves daily administration of allopurinol, to mitigate the adverse effects of the disorder. However, this medication is prone to side effects such as stomach upset, nausea, diarrhea, or drowsiness, and in relatively rare cases may cause more severe side effects, optionally even inducing very serious (possibly fatal) skin reactions. Therefore, there is a need for alternative treatment methods. According to some embodiments of the present invention, treatment of a subject having APRT deficiency may include administering a nano-carrier system comprising an adenine riboswitch encapsulated inside a VLP as disclosed herein.

In some embodiment, the metabolite is uric acid (urate), and said disorder is gout. Gout is a disorder caused by hyperuricemia (defined as serum urate higher than 6.8 mg/dL) that results in the precipitation of monosodium urate crystals in and around joints, most often causing recurrent acute or chronic arthritis. The initial attack (flare) of gout is usually monarticular and often involves the first metatarsophalangeal joint. Symptoms of gout include acute, severe pain, tenderness, warmth, redness, and swelling. Definite diagnosis requires identification of crystals in synovial fluid. Treatment of acute flares is with anti-inflammatory drugs. The greater the degree and duration of hyperuricemia, the greater is the likelihood that gout will develop. Urate levels can be elevated because of decreased renal (most common) or gastrointestinal excretion, increased production, or increased purine intake (usually in combination with decreased excretion). Gout may be developed as an IEM when the increased production occur as a primary hereditary abnormality, for example deficiency of hypoxanthine-guanine phosphoribosyltransferase (HPRT). Complete HPRT deficiency is defined as Lesch-Nyhan syndrome. According to some embodiments of the present invention, treatment of a subject having hyperuricemia, Lesch-Nyhan syndrome or gout may include administering a nano-carrier system comprising a xanthine riboswitch I encapsulated inside a VLP as disclosed herein.

Other examples of IEM include disorders in which the metabolite is at least one amino acid residue, any derivative, or any intermediate product or metabolite thereof, e.g. phenylalanine, tyrosine, glycine, homocysteine, arginine, cysteine, isoleucine, leucine, lysine, methionine, proline, tryptophane, valine, N-acetylaspartate (NAA), homogentisic acid, or derivatives thereof. For example, an IEM disorder associated with accumulation of Phenylalanine or derivatives thereof is Phenylketonuria, an IEM disorder associated with accumulation of Tyrosine or derivatives thereof is Tyrosinemia, an IEM disorder associated with accumulation of Glycine and derivatives thereof is Glycine encephalopathy, and an IEM disorder associated with accumulation of Homocysteine or derivatives thereof is Homocystinuria, and the IEM disorder associated with accumulation of at least one of isoleucine, leucine, valine and derivatives thereof is Maple Syrup Urine Disease (MSUD). In another embodiment an IEM associated accumulation of glutamine is glutaminase (GLS) deficiency.

In some particular embodiments of the invention, the methods of the invention are utilized for the treatment of glycine encephalopathy. Glycine encephalopathy is an autosomal recessive IEM with neurological effects at varying degrees, although in most cases the symptoms are very severe, with subjects often dying at early infancy. The cause of glycine encephalopathy is typically dysfunction of the glycine cleavage system (“GCS”), made up of four different protein subunits, due to a mutation in one of the genes encoding for a respective subunit. Dysfunction of the GCS leads to accumulation of glycine in blood plasma and cerebrospinal fluid, which is considered to be responsible for the severe symptoms of glycine encephalopathy. Treatment with a combination of sodium benzoate and dextromethorphan has been found to partially improve neurocognitive outcome, but the remedy is still far from complete. Therefore, there is a need for alternative treatment methods. According to some embodiments of the present invention, treatment of a subject having glycine encephalopathy may include administering a nano-carrier system comprising a glycine riboswitch encapsulated inside a VLP as disclosed herein.

In other embodiments, the systems and methods of the invention are used in the treatment of abnormally elevated metabolite levels in a subject in need thereof. In various embodiments, treatment of abnormally elevated metabolite levels (also referred to as “treating elevated metabolites”) may involve reducing the level of the metabolite in the subject, or in a cell, organ or bodily fluid thereof. In a particular embodiment, the use comprises reducing the circulating levels of said metabolites (typically in the blood serum). Advantageously, for treatment of abnormally elevated metabolite levels in the subject's blood or serum, a VLP carrier devoid of exogenously added targeting motifs may be used.

In some embodiments, the metabolite is vitamin B12. In another embodiment said subject is afflicted with, or is at risk for developing, hypervitaminemia B12. In other embodiments the metabolite is fluoride. In another embodiment said subject is afflicted with, or is at risk for developing, fluoride toxicity (e.g. due to ingestion of high fluoride quantity). In another embodiment the metabolite is uric acid. In another embodiment said subject is afflicted with, or is at risk for developing, hyperuricemia, Lesch-Nyhan syndrome or gout. Hyperuricemia is a condition of elevated levels of uric acid in the blood, which may lead to gout disease, creation of renal stones, and/or Lesch-Nyhan syndrome. Hyperuricemia can be due to over-production or consumption of uric acid, e.g., from natural excess production of purines (genetic reasons) or ingesting a purine-rich diet; or due to reduced release of uric acid from the body, e.g., due to kidney malfunctioning.

Additional examples of elevated metabolite conditions include e.g. APRT (adenine accumulation), ADA-SCID (adenine and/or deoxy-adenosine accumulation), xanthine oxidase deficiency (xanthine accumulation), gout (uric acid accumulation) Lesch-Nyhan syndrome (uric acid accumulation), phenylketonuria (phenylalanine accumulation), tyrosinemia (tyrosine accumulation), Mitochondrial neurogastrointestinal encephalomyopathy or MNGIE (thymidine accumulation), Purine nucleoside phosphorylase deficiency or PNP-deficiency (guanosine and/or dGTP accumulation), argininemia or arginase deficiency (arginine and/or ammonia accumulation), cystinuria, cystinosis (cysteine accumulation), alkaptonuria (homogentisic acid accumulation), glycine encephalopathy (glycine accumulation), iminoglycinuria (glycine, proline and/or hydroxyproline accumulation), hyperlysinemia (lysine accumulation), hypermethioninemia (methionine accumulation), homocystinuria (methionine and/or homocysteine accumulation), Canavan disease (N-acetylaspartate accumulation), hyperprolinemia (proline accumulation), hypertryptophanemia, hartnup disease (tryptophan accumulation), dihydropyrimidine dehydrogenase deficiency (thymine and/or uracil accumulation), Maple syrup urine disease (isoleucine, leucine, valine accumulation), isovaleric acidemia (isovaleric acid accumulation), isobutyryl-CoA dehydrogenase deficiency (valine accumulation), renal stones (e.g., nephrolithiasis, urolithiasis; calcium, oxalate, uric acid, and/or cystine accumulation), and cholelithiasis (cholesterol, bilirubin accumulation). In some embodiments, elevated blood metabolite conditions include elevation of an alpha hydroxy acid (e.g. lactate or 2-hydroxybutyrate), a carboxylic acid (e.g. Pro, Phe, Tyr, Ile, Leu, Val, Ala, Glu, Cystine, Gly, Gln, alphaaminoapidate or isocitrate) or a short-chain keto acid (e.g. Oxoisopentanoate or Pyruvate).

In other embodiments, the systems and methods of the invention are used in inhibiting or delaying the formation of metabolite assemblies in a subject in need thereof. The term “metabolite aggregates” or “metabolite assemblies” as used herein relates to accumulation of the specific metabolite in the cell in a specific fibrillar structures. More specifically, metabolite aggregates refer to well-ordered assembly or elongated nanoscale fibrillary structures of non-protein entities such as metabolites. These supramolecular fibrillar structures are formed via self-association of entities that accumulate in a cell. The formation of these fibrils, may resemble to amyloid-like fibrils and is typically described by a nucleation-dependent polymerization mechanism, which comprises nucleation and elongation, and is often considered as a kind of crystallization. It should be noted that in some embodiments, metabolite aggregates may be also referred to herein as amyloid-like structures or metabolite amyloids. Metabolite amyloids (e.g. adenine, tyrosine and phenylalanine) may play a key role in the development of metabolic disorders, as disclosed herein.

In some embodiments, accumulation of the metabolite and/or formation of metabolite aggregates may conveniently be determined using at least one of metabolic profiling, microscopy, light diffraction, absorption or scattering assay, spectrometric assay, immunological assay, Nuclear magnetic resonance (NMR), Liquid Chromatography, flow cytometry, and stereoscopy, by methods known in the art.

In particular, the systems and methods of the invention may be used in some embodiments for inhibiting (reducing the quantity) or delaying the (onset of) formation of non-proteinaceous metabolite assemblies, such as those formed of single amino acids and nucleobases, which assemblies may induce apoptotic cell death.

It is to be understood, that the nano-carrier system to be used in the methods of the invention is selected such that the functional RNA is capable of binding a metabolite associated with the disorder or condition in question, such that the disorder or condition (e.g. metabolic disorder, elevated metabolite levels or metabolite assemblies) is treated. Thus, for example, when the subject is afflicted with elevated adenine levels (e.g. in the blood), with the formation of adenine assemblies, and/or with a metabolic disorder associated therewith (e.g. APRT), the pharmaceutical composition to be administered to the subject may advantageously include a nano-carrier system, comprising an adenine riboswitch encapsulated inside a MS2 VLP. In another example, when the subject is afflicted with elevated levels of uric acid (e.g. hyperuricemia), with the formation of urate assemblies (e.g. kidney stones), and/or with a metabolic disorder associated therewith (e.g. Lesch-Nyhan syndrome or gout), the pharmaceutical composition to be administered to the subject may advantageously include a nano-carrier system, comprising a xanthine riboswitch I encapsulated inside a MS2 VLP.

The subject to be treated by the compositions and methods of the invention, also referred to herein as a patient in need thereof, is a mammalian and preferably a human subject. In certain embodiments, the subject has been diagnosed as suffering from a disorder or condition as disclosed herein. In other embodiments, said subject is diagnosed as being predetermined to or at risk for developing a disorder or condition as disclosed herein (e.g. due to a genetic predisposition).

### Food Technology

Cultured meat (CM) production is one of the most important components of the food technology industry to date, and is predicted to become the dominating technology for meat replacement. The technology is based on the controlled growth of animal-derived muscle cells in bioreactors and their processing into structures that resemble currently used meat. Despite the great potential in this field, the utilization of in vitro cell culturing of animal cells has several limitations which currently prohibit mass manufacture of cultured meat products. One of the most significant limitations is the formation of toxic byproducts as part of the cultivation process. Cell growth is associated with the consumption of carbon sources, amino acids, vitamins and other essential nutrients and the production of byproducts (metabolites) such as lactate and ammonia. Accumulation of excess lactate and ammonia in the culture medium is toxic and inhibitory for mammalian cell cultures. Current methods for removal of contaminating molecules include use of specifically engineered antibodies, which is not economically viable in the context of CM cell culture, and various filtration methods, which are not applicable for small molecules such as metabolites.

Thus, in some embodiments, the systems and methods of the invention are used for reducing the levels of a metabolite in a food product (e.g. a cultured meat food product). In some embodiments, the method comprises contacting the product or a precursor thereof with a chelating composition comprising a nano-carrier system, the system comprising a VLP, and a metabolite-specific functional RNA encapsulated inside the VLP, wherein the functional RNA maintains its ability to selectively bind the metabolite while being encapsulated inside said VLP, and is protected from nuclease degradation in vivo.

It is to be understood, that contacting the product or intermediate thereof with the chelating composition does not necessitate a physical contact between the product or intermediate and said composition. For example, contacting the product or intermediate with the composition is conveniently performed by supplementing said composition to the culture medium used to manufacture said product, which may be conveniently removed thereafter by conventional methods such as centrifugation, filtration and/or dialysis.

In addition, the contacting is performed such that the nano-carrier system or components thereof do not penetrate the cell membranes and do not enter the food product or intermediate. For example, the VLPs to be used in these embodiments do not contain a heterologous targeting sequence directed to the cultured cells. Thus, systems and methods of the invention may be used for the removal of excess metabolites while maintaining adequate safety for human consumption.

In another embodiment the product is in the form of aggregated particles of meat or textured plant protein. In another embodiment said product is a processed meat or meat analog product. In a particular embodiment said product is a CM product. In another embodiment said product is selected from the group consisting of patties, sausages, burgers, meatballs, cutlets, and hotdogs, wherein each possibility represents a separate embodiment of the invention. In another embodiment the intermediate is uncooked. In another embodiment said intermediate is in the form of an animal cell culture. In a particular embodiment said intermediate is in the form of a CM cell culture.

In yet other embodiments, said metabolite is selected from the group consisting of lactic acid, ammonia and ionic forms thereof (including lactate and ammonium). Thus, the metabolite-specific functional RNA may contain a synthetic riboswitch or metabolite-binding (aptamer) domain directed to lactic acid, lactate ammonia and ammonium, respectively.

Suitable methods for generating the aptamer domains have been described in the art, and employ, for example, “Systematic Evolution of Ligands by Exponential Enrichment” (“SELEX™”) described in, e.g., U.S. Pat. Nos. 5,475,096, and 5,270,163. The SELEX process is a method for the in vitro evolution of nucleic acid molecules with highly specific binding to target molecules. Douaki et al (Biosensors 2022, 12, 574, incorporated herein by reference) exemplify the generation of ammonium-binding RNA domains using a modified SELEX process employing machine learning and bioinformatics tools.

Thus, in another embodiment, there is provided a method of reducing the levels of at least one metabolite selected from the group consisting of lactic acid, ammonia and ionic forms thereof, in a cultured meat food product, comprising: admixing in culture media used to produce said food product a chelating composition comprising a nano-carrier system, the system comprising a MS2 VLP, and a metabolite-specific functional RNA encapsulated inside the VLP, wherein the functional RNA is specific to said at least one metabolite.

### Additional Embodiments

Despite the natural ability of viruses to protect their nucleic acid content in relatively inhospitable biological environments, the properties of synthetic engineered nano-particles derived from viral components, are yet to be characterized with respect to the particular payload in question. The present inventors engineered a new inventive nano-carriers technology, enabling the encapsulation and protection of functional RNA inside MS2 bacteriophage virus-like particles (VLPs). The present inventors identified the robust potential of RNA as a treatment of disease and various applications of administering RNA nanostructures to a subject to modify, reduce, or treat a disease in a subject.

The inventors recently engineered E. coli to produce MS2 VLPs encapsulating functional single strand DNA (ssDNA) by replacing the TR-RNA with an analogues DNA sequence, conjugated to the structural DNA sequence. The present invention in embodiments thereof discloses the use of a genetically engineered E. coli to produce MS2 VLPs encapsulating functional RNA in the form of RNA riboswitch, by conjugating the RNA riboswitch sequence to the TR-RNA sequence and co-expressing the MS2 coat proteins (CP) in vivo, resulting in the self-assembly of the VLPs nano-carriers within living bacteria. As described below, the VLPs nano-carriers maintained the riboswitch functionality and its ability to bind to specific molecules and metabolites. The VLP nano-carriers protected their RNA content from nucleases, thus overcoming the most significant limitation of RNA functionality in a nuclease rich environment. The present invention further demonstrates the VLP nano-carriers ability to act as a disease modifying treatment by applying them on an in vivo yeast model of adenine accumulation. This newly developed synthetic biology technology provides for the integration of state-of-the-art RNA nanotechnology into practical, potentially life-changing medical applications.

Such applications involve in some embodiments using RNA nanotechnology to modify, treat, or reduce levels of disease in a subject. In certain applications, the RNA nanotechnology forms a disease modifying treatment. The system is used to deliver functional RNA to treat abnormal levels of metabolites in disease. In certain embodiments, the VLP is a porous carrier (as analogous to a nano-gel), that is utilized for carrying functional RNA where abnormal level of metabolites are found. In certain embodiments, the functional RNA is targeted and performs various actions in order to modify, reduce, or treat a disease in a subject. In certain embodiments, objects of the invention are achieved by providing nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP. In certain embodiments, said VLP comprises a wild type Enterobacteria phage MS2 VLP. In certain embodiments, said functional RNA comprises RNA riboswitch. In certain embodiments, said VLP further comprises a packing sequence.

Other objects of the invention are achieved by providing a method of treating an elevated metabolites in a subject, comprising administering a treatment composition to the subject, wherein the treatment composition comprises a nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP. Other objects of the invention are achieved by providing a method of treating a metabolic disorder in a subject, comprising administering a treatment composition to the subject, wherein the treatment composition comprises a nano-carrier system comprising: a VLP, and a functional RNA encapsulated inside said VLP, wherein said VLP is configured to protect said functional RNA from nuclease degradation in vivo, and wherein said functional RNA maintains its functionality while being encapsulated inside said VLP. Other objects of the invention are achieved by providing a method of treating a metabolic disorder in a subject, comprising administering a treatment composition to the subject, wherein the treatment composition comprises a nano-carrier system comprising: a VLP made of up functional RNA, wherein aid functional RNA in said VLP is protected from nuclease degradation in vivo, and maintains its functionality.

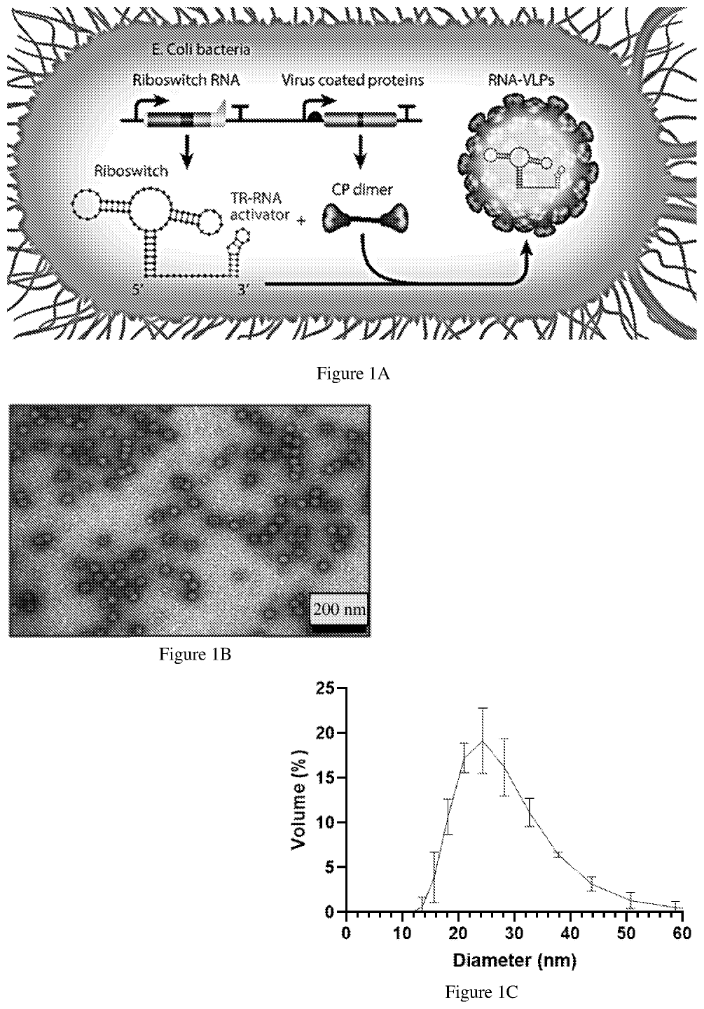

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention. In the following description, numerous details are set forth for the purpose of explanation. However, one of ordinary skill in the art will realize that the invention may be practiced without the use of these specific details. The present invention comprises an in vivo technology to produce functional RNA inside VLPs. These nanocarriers meet the requirements of applying RNA nanotechnology under real-world conditions. The present invention also comprises methods of treatment of using, administering, or applying functional RNA inside VLPs in order to modify, treat or reduce disease in subjects in need thereof. The RNA technology forms a disease modifying treatment, and systems and methods are used to deliver functional RNA to treat abnormal levels of metabolites in disease. In certain embodiments, the invention includes RNA being encapsulated in a VLP. The invention includes a VLP as a porous carrier (functionally analogous to a nano-gel), that is utilized for carrying functional RNA where abnormal level of metabolites are found. The invention further includes functional RNA that is targeted and performs various actions in order to modify, reduce, or treat a disease in a subject. Thus, disclosed in embodiments of the invention is the development of a disease-modifying treatment that disrupts the early stages of the metabolite self-assembly process, which is linked to metabolism disorders. As will be described in the Examples section below, a functional riboswitch was encapsulated inside MS2 bacteriophage VLPs (as illustrated in FIG. 1A). Utilizing the VLP as the nano-carrier (NC) of the riboswitch was unexpectedly found to provide three important features: (i) retaining the structure and associated function of the riboswitch, (ii) the VLPs pores permitted the passive influx of small molecules, such as metabolites, and (iii) the VLPs shell acted as a protective cage against nucleases, thus overcoming one of the most significant limitations of functional RNA applications.

The following examples are presented in order to more fully illustrate some embodiments of the invention. They should, in no way be construed, however, as limiting the broad scope of the invention.

### Examples

### Materials and Methods

Plasmid preparation: maturation protein and coat protein (CP) dimer were synthesized (IDT) as gene blocks and transformed into pACYC-DUET using Golden Gate standard procedure. The non-coding RNA sequence was inserted using reverse PCR (primers were purchased from IDT).

Induction and expression of VLP-Riboswitch nano-carries: the plasmid was transformed into E. coli BL21 (DE3) cells (Novagene), according to the manufacturer's instructions. Bacterial cells were grown in LB broth Miller (Difco) containing Chloramphenicol (25 μg/mL) (Gold Biotechnology, USA) at 37° C. to OD600=1.7. 10 mL of the bacterial culture was diluted in 1 L Terrific broth (TB, BactlLAB) containing chloramphenicol 25 g/mL, (1:1000) and cultivated at 37° c. to OD 600=0.4-0.8. Protein expression was induced by addition of 1 mM isopropyl-L-thio-D-galactopyranoside (IPTG) (Gold Biotechnology) at 37° c. and after 35 minutes, Rifampicin (Gold Biotechnology) was added to a final concentration of 200 μg/mL. The culture was further cultivated at 37° C. for 16 hours. The cell suspension was centrifuge at 4000 g for 20 minutes at 4° C. and cells were washed once in 200 mL PBS buffer (Hylabs) and centrifuged at 4000 g for 20 minutes in 4° C. The pellet was resuspended in 4 mL sonication buffer (100 mM NaH₂PO₄, 600 mM NaCl, pH 8.0, all from Sigma-Aldrich, complete EDTA-free protease inhibitor (Roche), 200 U of DNase I, and 200 μL of 10 mg/mL RNase A (both Thermofisher)). The cells were then lysed using ultrasonic disruption for a total of 120 seconds, 20% amplitude (Disintegrator Sonicator W385). To eliminate cell debris, the lysed bacterial suspension was centrifuged at 12000 RPM for 15-30 minutes at room temperature. Supernatant containing VLP-riboswitch nano-carriers was filtered through a 0.22-μm syringe filter (MERCK).

Purification of VLP-Riboswitch nano-carries using Ni bead columns: filtered supernatant was mixed in 1:1 ratio with 2×concentrated binding buffer (100 mM NaH₂PO₄, 600 mM NaCl, 30 mM imidazole, pH=8.0, all Sigma-Aldrich). Subsequently, His-tagged VLP-riboswitch nano-carries were purified using a Ni beads gravity column loaded with high-performance Ni beads (GE Healthcare). In brief, the column was pre-equilibrated with 50 mL of binding buffer (50 mM NaH₂PO₄, 300 mM NaCl, 15 mM imidazole, pH 8.0, all Sigma-Aldrich). The His-tagged VLP-riboswitch nano-carries bound to the column were subsequently washed with 100 mL washing buffer (50 mM NaH₂PO₄, 300 mM NaCl, 30 mM) from the column in a final volume of 10 mL with elution buffer (50 mM NaH₂PO₄, 300 mM NaCl, 200 mM imidazole, pH 8.0, all Sigma-Aldrich). All the eluted fractions were tested using a Bradford reagent assay (1:5 with ddH₂O) (190 μL Bradford, 10 μL sample, Bio rad). Positive fractions were desalted using 100 kDa amicons (MERCK) and converted first to UPW and then to riboswitch stabilizing buffer (10 mM MgCl₂ (MERCK), 50 mM Tris-HCl, pH 8.3 (Bio-Lab), and 100 mM KCl (MERCK)) for 24 hours each.

VLP-Riboswitch nano-carries protection against nucleases: VLP-riboswitch nano-carries as well as naked RNA (in vitro transcript) were incubated with and without 10 U of RNase A (Thermofisher) at 37° C. for 1 hour. Following incubation the samples were loaded onto 1% agarose gel electrophoresis).

In vitro transcription: a transcript of 1000 nucleotides was transcribed using the Riboprobe Combination System SP6/T7 RN (Promega), according to the manufacturer's instructions. The transcript was then purified using the MEGAclear™ Transcription Clean-Up Kit (Thermofisher scientific).

Yeast growth assays: the strains used in this work were BY4741 (WT) and the adenine salvage mutant, aah1Δ apt1Δ, that was established by a disruption of both AAH1 and APT1. The strains were grown overnight at 30° C. in synthetic defined SD medium (6.7 g/L Yeast Nitrogen Base without Amino Acids (Difco) and 20 g/L glucose (Merck)) containing a specific mixture of amino acids and nucleobases (without adenine). For OD600 measurements, strains were diluted to OD600=0.01. 200 μL of cells were platted on 96 wells microplates (Greiner, F-BOTTOM) with increasing concentrations of adenine (1, 2 and 4 mg/L) and with 2.5% (V/V) of the VLP-riboswitch nano-carries and incubated at 30° C. for 25 h with continuous shaking. OD600 was measured using Tecan™ SPARK 10M plate reader. The results displayed are representative of three biological experiments performed in triplicate.

Transmission Electron Microscopy (TEM): TEM imaging was performed by applying 10 μL samples onto 400-mesh copper grids covered by a carbon-stabilized Formvar film (SPI, West Chester, PA, USA). The samples were allowed to adsorb for 2 min before excess fluid was blotted off. Negative staining was then achieved by depositing 10 μL of 2% uranyl acetate on the grid for 2 min before blotting off excess fluid. Micrographs were recorded using a Tecnai 12 electron microscope (FEI, Tokyo, Japan) operating at 120 kV.

Dynamic Light Scattering Measurements: 1 mL of purified VLP-riboswitch nano-carries were taken. Particle sizes measured using Malvern Panalytical Zetasizer Nano ZS (Light Source: He—Ne laser 633 nm, Max 5 mW) with a protocol specified for protein in water solution. Measurements of a sample were done in triplicate in succession, and the reported values were the calculated Z average diameter and peak 1 average with standard deviation as error bars.

Flow cytometry: one milliliter of logarithmic cells incubated with adenine (2 mg/L) and VLPs (2.5% (V/V) were washed with PBS buffer and sonicated using 15 s pulses at 20% power. For each sample, 2*10⁶ cells were resuspended with ProteoStat dye (Enzo Life Sciences) diluted 1:3000 in ProteoStat assay buffer. Cells were incubated for 15 min at room temperature protected from light. Flow cytometry was performed using Stratedigm S1000EXi and the CellCapTure software (Stratedigm, San Jose, CA). Live cells were gated (P1) by forward scatter and side scatter. Fluorescence channels for FITC (530/30) and PE-Cy5 (676/29) were used utilizing a 488 nm laser source. A total of 50,000 events were acquired for each sample. Analyses were performed using FlowJo software (TreeStar, version 10). The results displayed are representative of three biological experiments performed in triplicate.

Confocal images: one milliliter of logarithmic cells incubated with adenine and VLPs (as mentioned above) was washed with PBS buffer and sonicated using 15 s pulses at 20% power and resuspended in 50 μL of ProtesoStat dye diluted 1:250 in PBS. Cells were incubated for 15 min at room temperature protected from light. 10 μL of each sample was deposited on poly-lysine-coated glass slides (Sigma-Aldrich). Cells were imaged using Leica TCS SP8 laser confocal microscope with ×63 1.4 NA or ×100 1.4 NA oil objectives. An argon laser with 488 excitation line was used for ProteoStat (emission wavelength, 500-600 nm). The results displayed are representative of three biological experiments.

### Example 1. Formation of Riboswitch-Encapsulating VLPs in Bacteria

A double-expression plasmid was synthesized, as described in “Materials and Methods” section above, encoding: (i) a MS2 CP dimer linked via HIS tag, and Maturation protein (“viral proteins”), and (ii) a non-coding RNA sequence, composed of the TR-RNA activator sequence in its 3′ conjugated to a functional RNA sequence is its 5′ (“Riboswitch RNA”). The sequences used for constructing the plasmid are set forth below (in the form of the corresponding sense DNA strand).

Adenine Riboswitch (A58U)-
(A58U riboswitch, SEQ ID NO: 1)
GCGCGTTGTATAACCTCAATAATATGGTTTGAGGGTGTCTACCAGGAACC
GTAAATTCCTGATTACAACGC.
TR-RNA-
(TR-RNA, SEQ ID NO: 2)
ACATGAGGATCACCCATGT.
Functional RNA construct including the
riboswitch and the TR-RNA (TMpac)-
(TMpac, SEQ ID NO: 3)
CGCTTCCGTCAAACCCCTGCGCGTTGTATAACCTCAATAATATGGTTTGA
GGGTGTCTACCAGGAACCGTAAATTCCTGATTACAACGCAAACCGGATGA
TAGACCTCACCTCCCCGCCCAATACTGAAATCTCATTAATACGCATACCC
CCACTATACACACGCAATCACCACATTAGCACAATGAATAATCATCGTAC
GGGAGAAAACATTCTAAACCCACATGAGGATCACCCATGT.
Coat protein (CP) Dimer (including CP1, CP2 and
a joining linker)-
(CP dimer, SEQ ID NO: 4)
AGCCCTCAACCGGAGTTTGAAGCATGGCTTCTAACTTTACTCAGTTCGTT
CTCGTCGACAATGGCGGAACTGGCGACGTGACTGTCGCCCCAAGCAACTT
CGCTAACGGGGTCGCTGAATGGATCAGCTCTAACTCGCGTTCACAGGCTT
ACAAAGTAACCTGTAGCGTTCGTCAGAGCTCTGCGCAGAATCGCAAATAC
ACCATCAAAGTCGAGGTGCCTAAAGTGGCAACCCAGACTGTTGGTGGTGT
AGAGCTTCCTGTAGCCGCATGGCGTTCGTACTTAAATATGGAACTAACCA
TTCCAATTTTCGCTACGAATTCCGACTGCGAGCTTATTGTTAAGGCAATG
CAAGGTCTCCTAAAAGATGGAAACCCGATTCCCTCAGCAATCGCAGCAAA
CTCCGGCATCTACGCTAACTTTACTCAGTTCGTTCTCGTCGACAATGGCG
GTACCCATCACCATCACCATCACGGTACCGGCGACGTGACTGTCGCCCCA
AGCAACTTCGCTAACGGGGTCGCTGAATGGATCAGCTCTAACTCGCGTTC
ACAGGCTTACAAAGTAACCTGTAGCGTTCGTCAGAGCTCTGCGCAGAATC
GCAAATACACCATCAAAGTCGAGGTGCCTAAAGTGGCAACCCAGACTGTT
GGTGGTGTAGAGCTTCCTGTAGCCGCATGGCGTTCGTACTTAAATATGGA
ACTAACCATTCCAATTTTCGCTACGAATTCCGACTGCGAGCTTATTGTTA
AGGCAATGCAAGGTCTCCTAAAAGATGGAAACCCGATTCCCTCAGCAATC
GCAGCAAACTCCGGCATCTACTAATAGACGCCGG.
Gblock gene fragment including Maturase and CP
Dimer coding sequences-
(maturase and CP dimer, SEQ ID NO: 5)
ACGACTCACTATAGGGGAATTGTGAGCGGATAACAATTCCCCTGTAGAAA
TAATTTTGTTTAACTTTAATAAGGAGATATACCATGGTGGCTATCGCTGT
AGGTAGCCGGAATTCCATTCCTAGGAGGTTTGACCTGTGCGAGCTTTTAG
TACCCTTGATAGGGAGAACGAGACCTTCGTCCCCTCCGTTCGCGTTTACG
CGGACGGTGAGACTGAAGATAACTCATTCTCTTTAAAATATCGTTCGAAC
TGGACTCCCGGTCGTTTTAACTCGACTGGGGCCAAAACGAAACAGTGGCA
CTACCCCTCTCCGTATTCACGGGGGGCGTTAAGTGTCACATCGATAGATC
AAGGTGCCTACAAGCGAAGTGGGTCATCGTGGGGTCGCCCGTACGAGGAG
AAAGCCGGTTTCGGCTTCTCCCTCGACGCACGCTCCTGCTACAGCCTCTT
CCCTGTAAGCCAGAACTTGACTTACATCGAAGTGCCGCAGAACGTTGCGA
ACCGGGCGTCGACCGAAGTCCTGCAAAAGGTCACCCAGGGTAATTTTAAC
CTTGGTGTTGCTTTAGCAGAGGCCAGGTCGACAGCCTCACAACTCGCGAC
GCAAACCATTGCGCTCGTGAAGGCGTACACTGCCGCTCGTCGCGGTAATT
GGCGCCAGGCGCTCCGCTACCTTGCCCTAAACGAAGATCGAAAGTTTCGA
TCAAAACACGTGGCCGGCAGGTGGTTGGAGTTGCAGTTCGGTTGGTTACC
ACTAATGAGTGATATCCAGGGTGCCTATGAGATGCTTACGAAGGTTCACC
TTCAAGAGTTTCTTCCTATGAGAGCCGTACGTCAGGTCGGTACTAACATC
AAGTTAAATGGCCGTCTGTCGTATCCAGCTGCAAACTTCCAGACAACGTG
CAACATATCGCGACGTATCGTGATATGGTTTTACATAAACGATGCACGTT
TGGCATGGTTGTCGTCTCTAGGTATCTTGAACCCACTAGGTATAGTGTGG
GAAAAGGTGCCTTTCTCATTCGTTGTCGACTGGCTCCTACCTGTAGGTAA
CATGCTCGAGGGCCTTACGGCCCCCGTGGGATGCTCCTACATGTCAGGAA
CAGTTACTGACGTAATAACGGGTGAGTCCATCATAAGCGTTGACGCTCCC
TACGGGTGGACTGTGGAGAGACAGGGCACTGCTAAGGCCCAAATCTCAGC
CATGCATCGAGGGGTACAATCCGTATGGCCAACAACTGGCGCGTACGTAA
AGTCTCCTTTCTCGATGGTCCATACCTTAGATGCGTTAGCATTAATCAGG
CAACGGCTCTCTAGATAGAGCCCTCAACCGGAGTTTGAAGCATGGCTTCT
AACTTTACTCAGTTCGTTCTCGTCGACAATGGCGGAACTGGCGACGTGAC
TGTCGCCCCAAGCAACTTCGCTAACGGGGTCGCTGAATGGATCAGCTCTA
ACTCGCGTTCACAGGCTTACAAAGTAACCTGTAGCGTTCGTCAGAGCTCT
GCGCAGAATCGCAAATACACCATCAAAGTCGAGGTGCCTAAAGTGGCAAC
CCAGACTGTTGGTGGTGTAGAGCTTCCTGTAGCCGCATGGCGTTCGTACT
TAAATATGGAACTAACCATTCCAATTTTCGCTACGAATTCCGACTGCGAG
CTTATTGTTAAGGCAATGCAAGGTCTCCTAAAAGATGGAAACCCGATTCC
CTCAGCAATCGCAGCAAACTCCGGCATCTACGCTAACTT.

The double expression plasmid was transformed to E. coli DE3 cells, for co-expression of the viral proteins and non-coding RNA, as described in “Materials and Methods” section above. The resulting viral particles (also referred to herein as RNA-VLPs or VLP-riboswitch nano-carriers) were purified and characterized by transmission electron microscopy (TEM) and dynamic light scattering (DLS). A schematic illustration of the VLP production system is provided in FIG. 1A, and the TEM and DLS analyses are presented in FIGS. 1B-1C, respectively.

In FIG. 1B is shown a TEM image of the in vivo produced VLP-RNA nano-carriers (produced in bacterial cells, scale bar: 200 nm), and in FIG. 1C is presented a DLS measurements of the VLP-riboswitch nano-carriers. As can be seen in FIGS. 1B and 1C, the VLP-riboswitch nano-carriers were of a diameter of 25-30 nm. A particle population having a peak at about 25 nm, more specifically about 27 nm is shown in FIG. 1C. Thus, the diameter of the VLP-riboswitch nano-carriers produced by the system is similar to that of the wild-type (WT) MS2 capsids.

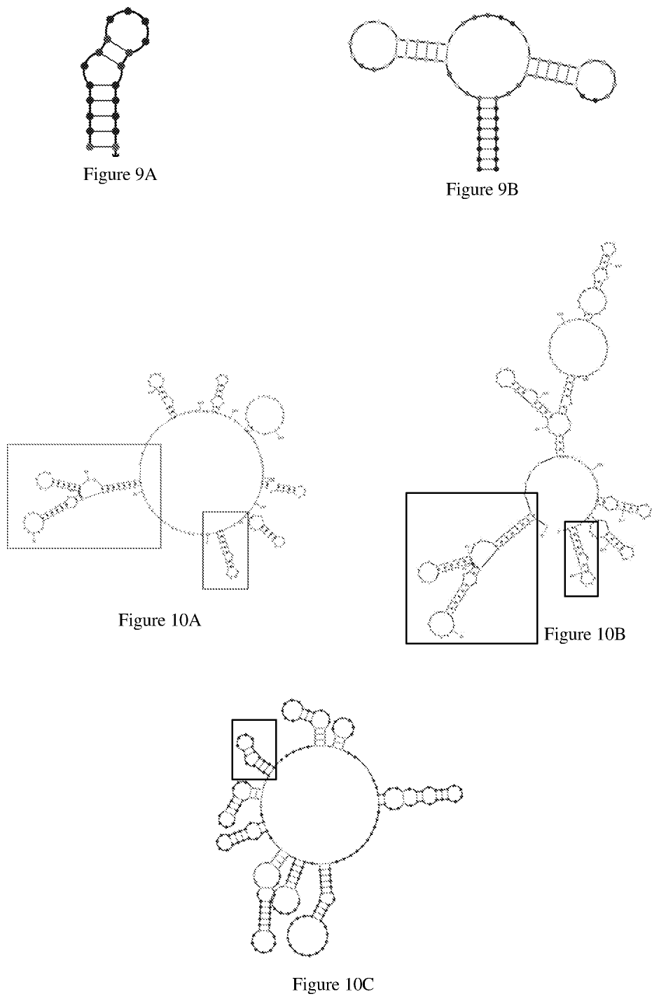

Thus, the co-expression of the MS2 CP dimer with the non-coding RNA containing the TR-RNA activator sequence resulted in the self-assembly of the MS2 VLP-riboswitch nano-carriers within a live organism. The functional secondary structure of the TR-RNA, as computed by NUPACK platform, is shown in FIG. 9A, and will be further described in Example 5. Without wishing to be bound by a specific theory or mechanism of action, encoding for the TR-RNA activator sequence at the 3′ of the non-coding RNA eliminated the potential encapsulation of non-desired RNA intermediates, as evident from the high yield of functional VLPs produced.

### Example 2. VLP Nano-Carriers Protect Encapsulated Riboswitch from Degradation

The stability of the VLP-RNA nano-carriers against nucleases was tested by their incubation with 10 U of RNase A (Thermofisher) at 37° C. for 1 hour. As a control for the reaction, naked RNA (an in vitro transcript, see Materials and Methods) was also incubated under the same conditions. As additional controls, VLP-RNA nano-carriers and naked RNA, respectively, were also incubated in a RNase-free medium at 37° C. for 1 hour. The ability of the VLP-RNA nano-carriers to resist the impact of nucleases, specifically RNases, was determined using agarose gel electrophoresis, as is shown in FIG. 2 (“VLPs”—treated VLP-RNA nano-carriers; “RNA”—treated naked RNA).

As can be seen in FIG. 2, the VLP-RNA nano-carriers were resistant under these conditions and were able to protect the encapsulated RNA. In contradistinction, the naked RNA rapidly degraded under the same conditions. In addition, in a semi-quantitative assay performed essentially as described above, at least 90-95% of the VLP-encapsulated RNA remained non-degraded in the presence of 10 U of RNase A at 37° C. for 1 hour, as well as in the presence of 10 U DNase I treatment for 24 h at 37° C. It should also be noted that during the purification process of the VLP-RNA nano-carriers from the bacteria, RNase and DNase were added in excess to the lysis buffer to eliminate endogenous RNA and DNA of E. coli, as described in “Methods and Materials” section above. This indicated that the VLP-RNA nano-carriers are also resistant to DNase.

RNA is highly prone to degradation under cellular conditions and, therefore, needs to be assembled into RNA nanoparticles or to be chemically modified to reduce its degradation rate. Remarkably, in the described technology, the VLPs' shell protected the RNA both under cellular conditions (inside living bacteria) and in the presence of RNase and DNase (during the purification process and in the experiments presented in FIG. 2). As a result, the encapsulated RNA did not require additional structural design or chemical modifications to increase its stability in vivo. The VLP nano-carriers, whose self-assembly within the live bacteria was driven by the TR-RNA activator sequence of the Riboswitch RNA itself, conveyed effective protection to the Riboswitch RNA.

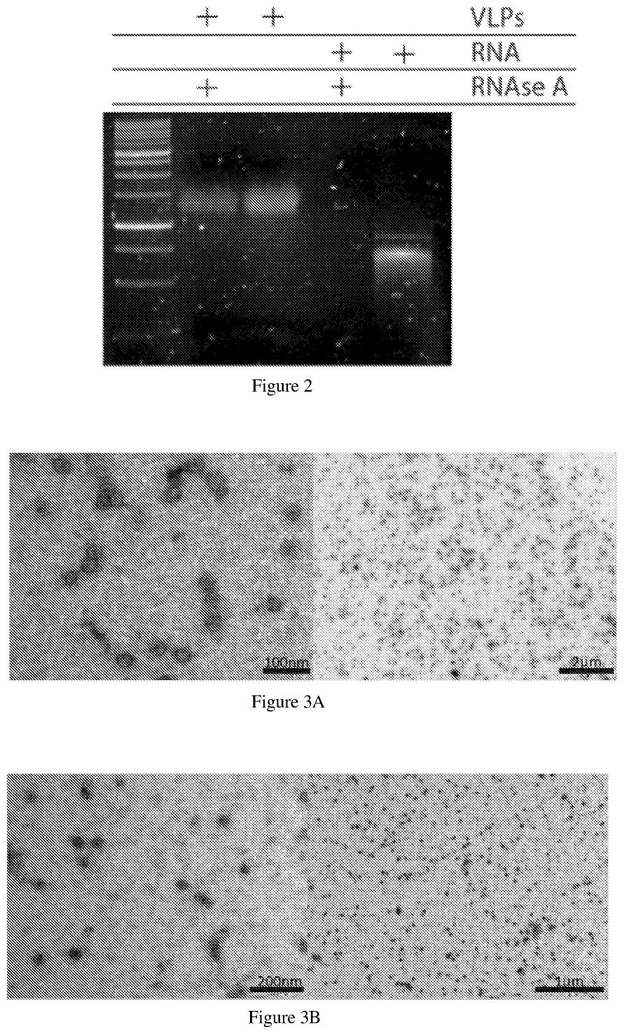

The VLP nano-carriers were further tested for long-term stability. The purified VLPs were preserved at 4° C. riboswitch buffer (defined above) and were periodically observed to assess their appearance at the following time points: 2 weeks, and 1, 2, 3, 6 and 9 months. FIGS. 3A and 3B are representative TEM images (each including two different scales, as denoted) of VLP riboswitch nano-carriers, at 2 weeks and 9 months after purification from the bacterial host, respectively. As can be clearly seen, the VLPs remained completely intact for at least nine months, as determined from the characteristic ˜25-30 nm diameter spherical particles, which were clearly viewed both at 2 weeks and at 9 months (and at all the intermediate intervals). This remarkable result may have considerable practical implications, as it indicates that the VLP-riboswitch nano-carriers may be utilized as a pharmaceutical composition with a relevant shelf life, being maintained for at least nine months in conventional refrigeration storage (4-8° C.).

In summary, the VLPs were found to exert exceptional protection of functional RNA against endonucleases in vivo, and to provide a highly effective protective shell for long-term maintenance of the encapsulated functional riboswitches.

### Example 3. Rescue Effect of VLP-Riboswitch Nano-Carriers as Compared to Rescue Effect of Tannic Acid in Excess-Adenine Yeast Model

Next, the effect of the VLP-riboswitch nano-carriers on the model yeast was compared to the effect of tannic acid (TA) thereupon. TA is a widely studied polyphenol with anti-amyloidogenic activity against β-amyloid in vitro, and its inhibitory effect on adenine both in vitro and in vivo was previously shown, using the same yeast model as described in Example 3 (aah1Δ apt1Δ). In the in vivo model, it was discovered that the addition of TA to the media resulted in a significant decrease of amyloid-like structures of the adenine inside the yeast and it is considered that the TA may be able to enter to the cell and inhibit the self-assembly process of the accumulated adenine.

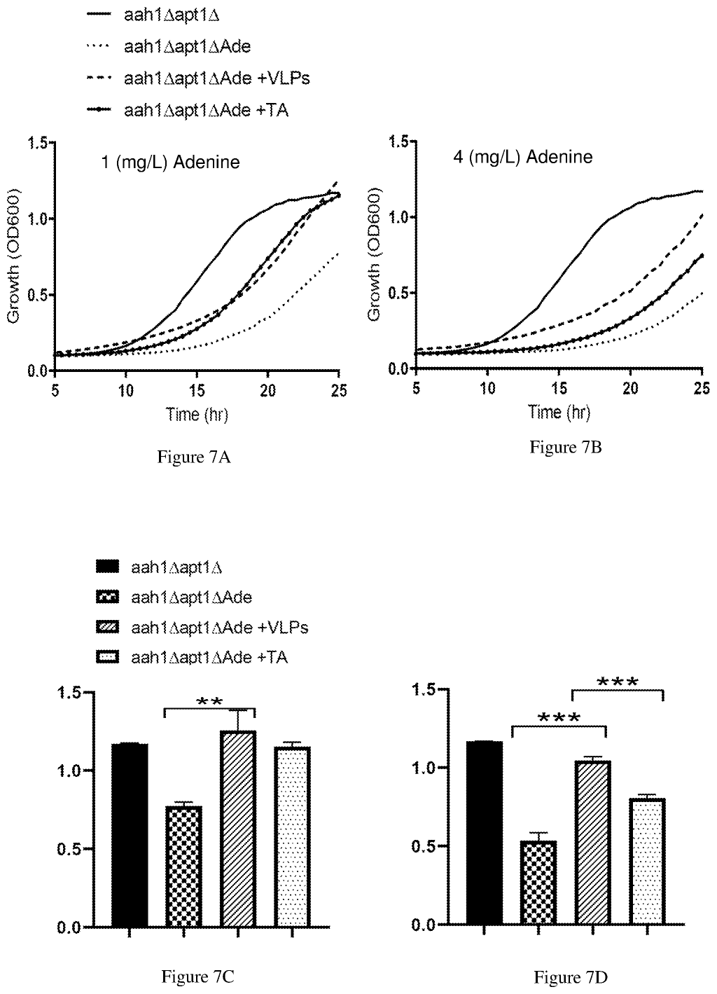

Similar to the assay described above (in Example 3), the model yeast aah1Δ apt1Δ was grown under different media conditions: (i) without adenine, (ii) with adenine, (iii) with adenine and VLP-riboswitch nano-carriers and (iv) with adenine and TA. As can be seen in FIGS. 7A through 7D, both in the low and high adenine concentrations (1 mg/L and 4 mg/L, respectively), the VLP-riboswitch nano-carriers and TA showed an improved growth of the model yeast. However, surprisingly, in the high concentration of adenine in the media (4 mg/L), the VLP-riboswitch nano-carriers significantly improved the growth even when compared to the growth improvement provided by TA (FIG. 7D).

As explained with reference to FIG. 5 above, the riboswitch VLPs are disclosed to perform an early intervention in the adenine accumulation process. In contradistinction, TA is considered to inhibit the self-assembly process of the accumulated adenine inside the yeast. Thus, without wishing to be bound by a specific theory or mechanism of action, the VLP-riboswitch nano-carriers interfere with the accumulation process a few steps earlier by preventing some of the adenine from entering the yeast, thereby leading to less accumulated adenine inside the yeast, which provides a superior and significantly enhanced phenotypic rescue.

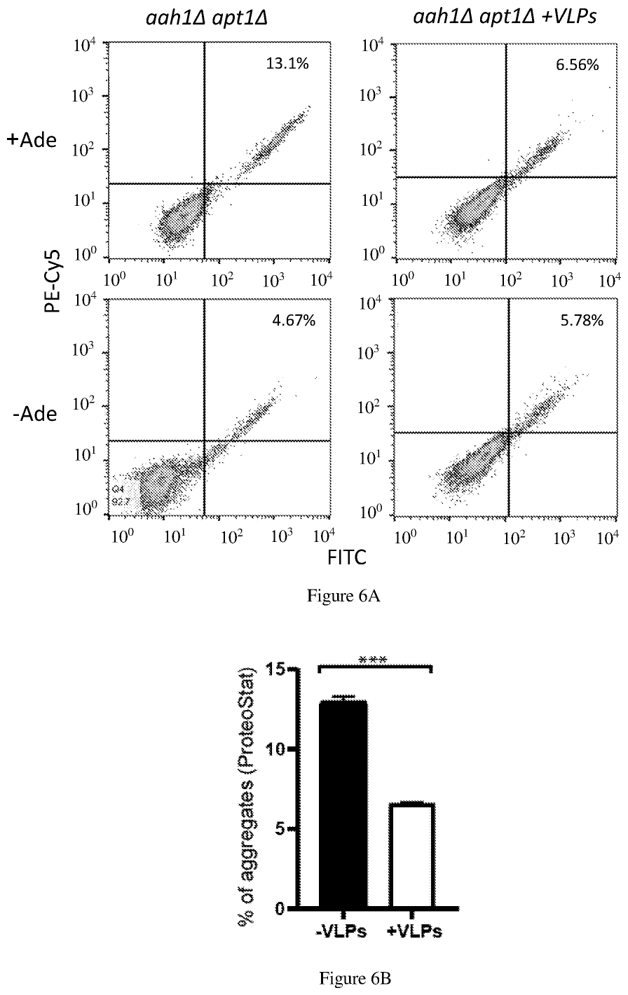

The effect of the supplementation of the growth media of aah1Δ apt1Δ yeast with the VLP-riboswitch nano-carriers on the formation of amyloid-like aggregates of adenine in the yeast, was further tested by flow cytometry. ProteoStat staining, which stains amyloid fibrils and aggregates, was used on the system to detect the presence of amyloid-like adenine aggregates in the model yeasts. The staining of the cells was analyzed by flow cytometry, as described in the “Materials and Methods” section above. The results are shown in FIGS. 6A-6B, in which the percent of cells with metabolite aggregation is represented by the dots which fall in the Upper Right quadrant (Q2) of the flow cytometry chart (due to the aggregate Proteostat staining which returns a higher fluorescent signal).

As is shown in FIGS. 6A and 6B, the percent of cells with metabolite aggregation significantly decreased in the presence of adenine and VLP-riboswitch nano-carriers, as compared to the cells with adenine and without the Riboswitch-VLPs. In particular, the results show a twofold reduction in the incidence of aggregates, from 13.1% to 6.56%. In a separate experiment, performed essentially as described above with a second experimental group of aah1Δ apt1Δ yeast cells, stained by Proteostat and subjected to flow cytometry analysis, the percent of cells displaying adenine aggregates when no adenine was added to the media was below 1%, while with added adenine was 7.83%. Supplementation of Riboswitch-VLPs reduced the incidences of adenine aggregates to 3.73%, displaying over a twofold reduction.

It is noted that as a control experiment, wild-type (WT) yeast cells with and without added adenine were subjected to a flow cytometry analysis (following Proteostat staining, essentially as described above). Both experimental groups displayed a substantially equal minimal level of adenine aggregates, namely ˜0.1%.

Confocal microscopy was also used to further identify the assembly and intracellular localization of the adenine aggregates, as described in “Methods and Materials” above. Stained dots (indicative of metabolite aggregates), were seen only in the mutant yeast model following the addition of adenine, and were not detected in WT yeast, or in mutant yeast in the presence of the Riboswitch-VLPs, reinforcing the indication of the preventive effect of the Riboswitch-VLPs on the formation of adenine aggregates within the aah1Δ apt1Δ yeast.

Thus, further to the highly substantial rescue effect of the Riboswitch-VLPs on the growth rate of the mutant yeast cells described above, it is herein demonstrated that the Riboswitch-VLPs significantly decrease adenine aggregation and corresponding accumulation of amyloid fibrils in a living organism. This is an additional indication that the Riboswitch-VLPs may serve as a possible disease modifying treatment for metabolic disorders, as in most of the disorders abnormalities arise due to the accumulation of metabolites, which are toxic or interfere with the normal function of cells and tissues.

In conclusion, the inventors were able to genetically engineer E. coli to produce MS2 VLPs encapsulating functional RNA. These VLPs can act as efficient nano-carriers capable to function under relevant biological conditions. The encapsulation of the functional RNA inside a protective shell holds great potential for diverse fields, including metabolic disorders, sensing, specific delivery to name a few. Most of the RNA nanotechnology applications are relevant to biological conditions outside-the-lab in a relatively inhospitable environment. Therefore, this new engineered nano-carriers can serve as one of the missing links in translating RNA nanotechnology to real-world application.

### Example 4. Rescue Effect of VLP-Riboswitch Nano-Carriers in Excess-Adenine Yeast Model

Following the successful encapsulation and protection of the riboswitches within the VLPs, the ability of the riboswitch to remain functional while enclosed within VLP-riboswitch nano-carriers was tested. The secondary structure of the functional riboswitch, as computed by NUPACK platform, is shown in FIG. 9B and will be further related to in Example 5. The inventors aimed to apply the VLP-RNA nano-carriers as a possible disease modifying treatment for metabolic disorders. To this end, a model yeast strain, denoted aah1Δ apt1Δ, was utilized, in which both adenine salvage genes APT1 and AAH1 were disrupted, leading to adenine accumulation. Adenine is part of the purine biosynthesis pathways, which are crucial to maintain the normal function of cells and are conserved between yeasts and humans. In humans, adenine salvage occurs using two enzymes; adenine phosphoribosyl transferase (APRT) and adenosine deaminase (ADA). Mutations in these enzymes can lead to adenine accumulation and its derivatives: mutation in APRT leads to APRT deficiency and mutation in ADA leads to ADA deficiency. In budding yeasts, the APRT and ADA orthologs are APT1 and AAH1, respectively, and they are involved in the adenine salvage pathway similar to APRT and ADA in humans.

Riboswitches are RNA-based genetic control elements that are usually found in non-coding regions of RNAs and can be found both in prokaryotes and eukaryotes. The riboswitches act as genetic switches, are highly structured, and are able to bind target cellular metabolites with high selectivity. VLP-RNA nano-carriers encapsulating functional RNA in the form of riboswitch that can specifically and selectively bind adenine, produced in E. coli as described in Example 1, were evaluated for their effect in this yeast model. The natural pbuE adenine riboswitch is able to selectively bind adenine with K_{d}=354±17 nM. The A58U riboswitch encapsulated in the VLPs is a modified riboswitch with a point mutation that increases the K_{d} to 273±17 nM, i.e., increases the affinity of the riboswitch to its adenine ligand (a decreased K_{d} represents a lower dissociation constant, i.e., a higher affinity).

The aah1Δ apt1Δ mutant yeast model was grown in media without adenine or in media with 1, 2, and 4 mg/L of adenine, respectively. For each adenine concentration, the media of one experimental group were supplemented with the Riboswitch-VLPs from T=0, and the growth of the aah1Δ apt1Δ with adenine and the VLP-riboswitch nano-carriers was compared to the growth of the control groups, namely aah1Δ apt1Δ with and without adenine.

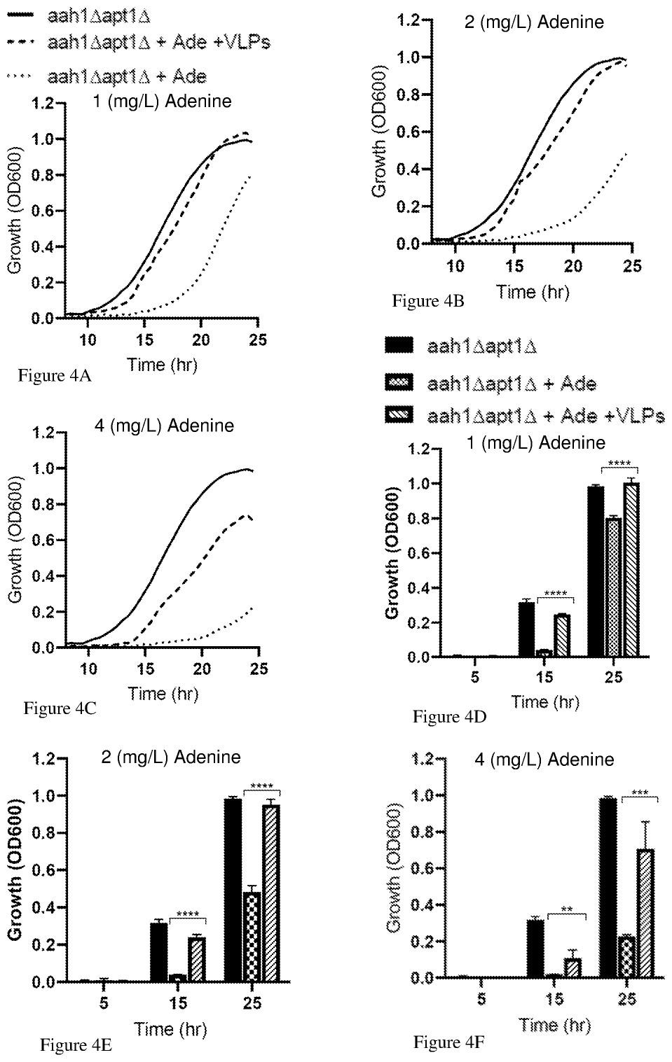

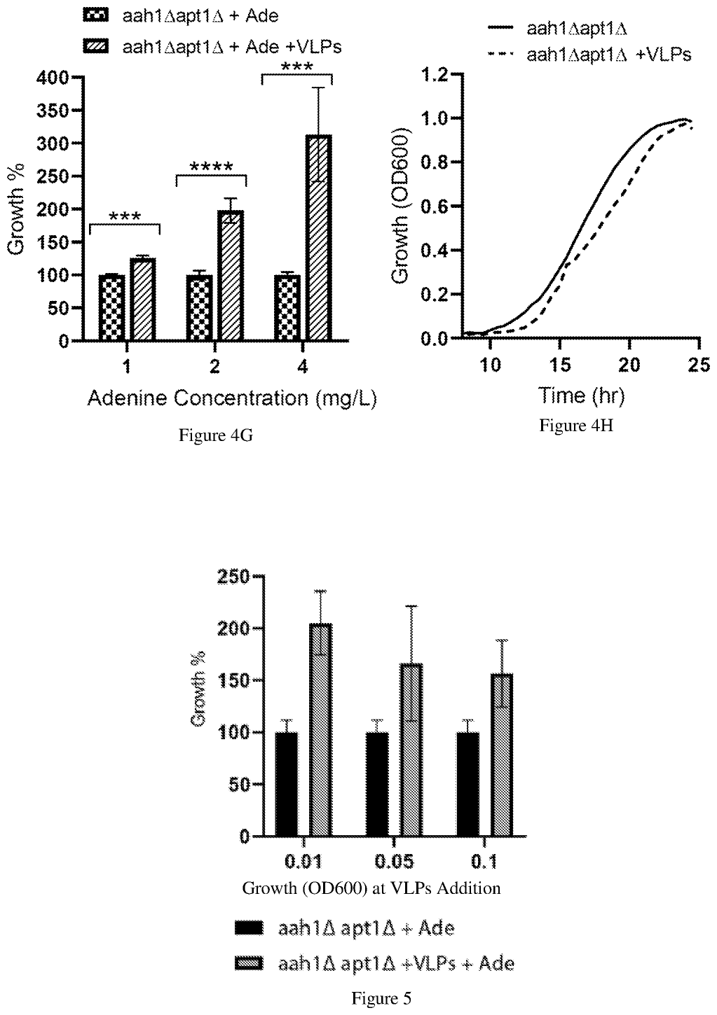

In FIGS. 4A-4C are shown the growth rates of aah1Δ apt1Δ mutant yeast populations as a function of time, at the respective different adenine concentrations (FIG. 4A—1 (mg/L), FIG. 4B—2 (mg/L), FIG. 4C—4 (mg/L)) without supplementary VLP-riboswitch nano-carriers (“aah1Δ apt1Δ+Ade”), or with addition of the VLPs (“aah1Δ apt1Δ+Ade+VLPs”), as compared to the growth rates of aah1Δ apt1Δ yeast population with no added adenine and no added VLPs (“aah1Δ apt1Δ”). FIGS. 4D to 4F correspond to FIGS. 4A to 4C, respectively, showing the growth rates of the different yeast populations (“aah1Δ apt1Δ”, “aah1Δ apt1Δ+Ade”, “aah1Δ apt1Δ+Ade+VLPs”) at particular time points (5, 15, and 25 hours).

As can be seen FIGS. 4A-4F, the aah1Δ apt1Δ yeast model grew at a slow rate on media containing adenine in comparison to media without adenine, and the growth rate deteriorated as the adenine concentration was elevated. However, remarkably, supplementing the media containing adenine with the VLP-riboswitch nano-carriers resulted in a highly significant rescue phenotype of the aah1Δ apt1Δ yeast model. The results indicate that despite being encapsulated in VLPs, addition of the functional RNA in the form of VLP-riboswitch nano-carriers to the media improved the growth of aah1Δ apt1Δ yeast significantly in comparison to aah1Δ apt1Δ growth without the VLPs (FIG. 4A-4F).

The magnitude of the rescue effect was further examined by comparing the relative percentage of growth (“Growth %”) with and without the VLP-riboswitch nano-carriers for each of the adenine concentrations (1, 2, and 4 mg/L), respectively (FIG. 4G). As can be seen, the rescue effect substantially increased in correlation to the increase of adenine concentration in the media, showing that the supplementation of the media with the VLP-riboswitch nano-carriers had the most dramatic effect in high adenine concentrations, namely up to 300% increase in yeast growth.

In most metabolic disorders, abnormalities arise due to the accumulation of metabolites, which are toxic or interfere with the normal function of cells and tissues. The present inventors had shown that several metabolites, including adenine, that are associated with metabolic disorders, can form ordered self-assembled amyloid like-structures in vitro, and demonstrated these assemblies' ability to induce apoptotic cell death. The inventors further established an in vivo model of adenine self-assembly in yeast, in which the yeast strain is blocked in the enzymatic pathway downstream to adenine, which led to toxicity that is associated with the intracellular accumulation of adenine. It is noted, that even under normal adenine concentrations, which are required for the healthy growth of WT yeast, the growth of the aah1Δ apt1Δ yeast model is hampered, relative to when no adenine is added. This corresponds to the toxicity of normal metabolite concentrations observed in some inborn error of metabolism disorders, such as phenylketonuria and tyrosinemia, where the required daily allowance for healthy population is actually toxic to patients, due to accumulation of the metabolites in the absence of salvage (Gazit et al., 2019). The results presented in FIGS. 4A-4G demonstrate some of the VLP-riboswitch nano-carriers features as a possible new disease modifying treatment. Surprisingly, the riboswitch inside the VLPs remained functional and was able to bind the adenine and deny access to it by the yeasts, thus protecting the mutant yeasts from the toxic adenine accumulation, while the VLPs acted as a protecting shell for the riboswitch against nucleases, thus increasing the riboswitch life-time and function.

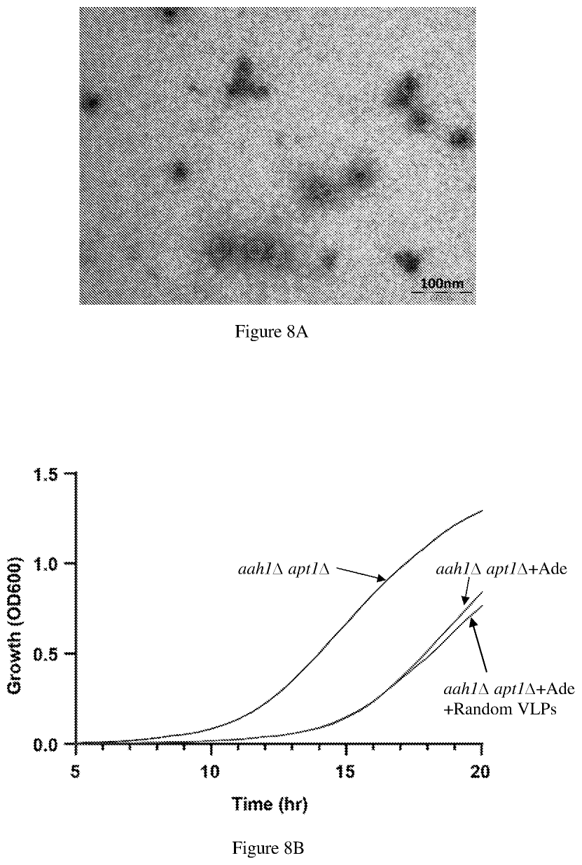

It is further noted that the addition of the VLP-riboswitch nano-carriers alone did not affect the growth of the aah1Δ apt1Δ, as shown in FIG. 4H. In addition, as presented in FIGS. 8A and 8B, when the growth media of the aah1Δ apt1Δ yeast was supplanted with VLPs encapsulating random RNA sequence instead of the riboswitch sequence, the VLPs had no rescuing effect on the yeast's growth. This emphasizes that the rescued phenotype of the yeasts results directly from the ability of the encapsulated riboswitch to bind adenine, and is not related to the mere presence of the entire VLP-RNA moieties.

The effect of supplementing the media with the Riboswitch-VLPs was further tested at different time points (i.e., different cell confluences, manifested as different OD600 values). As can be seen in FIG. 5, the Riboswitch-VLPs were found to be most effective at enhancing yeast growth when the VLPs were added at earlier time points of the yeast growth (OD600=0.01), while a less pronounced effect was observed when the VLPs were added at later stages (4E.g., OD600=0.1). This may suggest that the Riboswitch-VLPs are most effective before nucleation and early oligomerization of the metabolite inside the cells, which further indicates the Riboswitch-VLPs activity in binding the adenine in the media and preventing its intake by the yeast.

### Example 5. In Silico Analysis of RNA Structure and Functionality

Further to the experiments described above, in silico analyses were performed to evaluate the structure of various RNA constructs under different environmental conditions. To this end, several RNA constructs were designed, with alternative positions for the riboswitch and the TR sequence, and alternative spacer sequences, and evaluated in the NUPACK and OligoAnalyzer™ Tool platforms. Initially, the sequences of each of the isolated TR-RNA and RNA-riboswitch elements were analyzed independently, using the NUPAK platform (electing a temperature of 30° C.), and their resulting secondary structures are presented in FIGS. 9A and 9B, respectively. FIG. 9A shows that the TR-RNA activator sequence assumed its characteristic stem-and-loop formation associated with its reported function, and FIG. 9B shows the functional two-armed formation of the adenine riboswitch in its unbound conformation (i.e., the conformation which is operational to bind the adenine ligand). In additional analyses these structures were found to be maintained under the experimental conditions tested (corresponding to yeast and human systems, as detailed below) as determined by both platforms. These structures are further referred to below as the functional conformations (or functional structures) of the TR-RNA and riboswitch, respectively.

The structure of the encoded RNA construct used in the experiments described in Examples 1-4 was then analyzed. The analyses included the following: 1) evaluation under conditions characteristic of yeast growth (30° C.), and 2) evaluation under physiological conditions characteristic of human sera (37° C., and Na⁺ and Mg²⁺ concentrations of 135 mM and 0.8 mM, respectively).

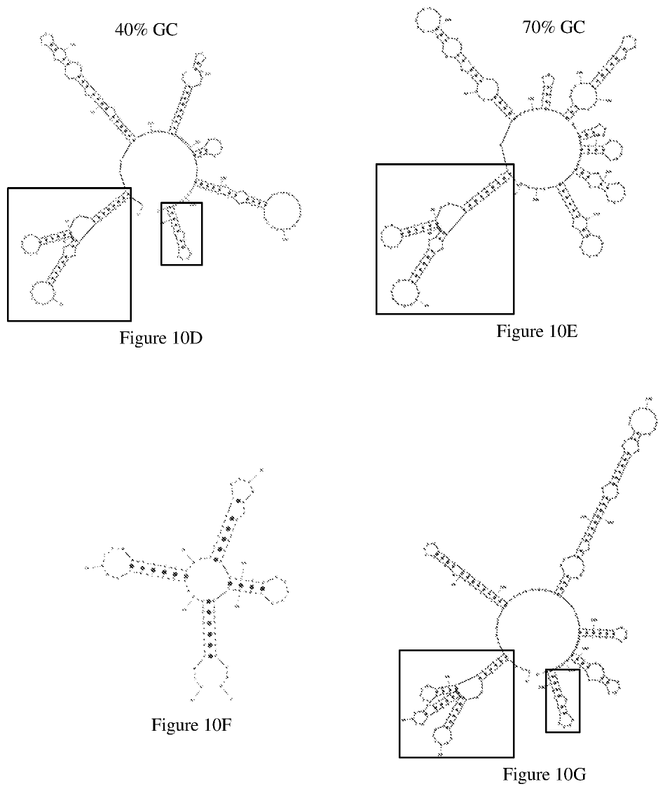

As can be seen in FIG. 10A, depicting the structural analysis produced by the OligoAnalyzer™ Tool of the RNA construct of SEQ ID NO: 6, both the TR-RNA and the riboswitch functional conformations (marked by surrounding boxes for clarity; left-box—riboswitch, right-box—TR-RNA activator) were formed under human sera conditions. The functional conformations of both elements appeared also when analyzed under yeast growth conditions.

The results show that the RNA construct, which procured the substantial growth rescue effect in the yeast model, displayed the functional RNA structures associated with driving the encapsulation of the RNA construct (TR-RNA) and with binding the adenine (RNA-riboswitch), which together brought about the rescue effect. In addition, the same functional structures that were formed in the yeast growth conditions were remarkably formed also in the human sera conditions. This indicates that the same adenine salvage effect could be achieved under physiological conditions characteristic of the human body, which could be highly useful for treating diseases related to excess metabolites in human patients.

The sequence of the functional RNA analyzed above, in which the sequences of the TR-RNA (inserted at position 222) is underlined and that of the riboswitch element (inserted at position 19) is marked in bold and underlined, is set forth in SEQ ID NO: 6 as presented below:

(19-position riboswitch RNA, SEQ ID NO: 6)
CGCUUCCGUCAAACCCCUGCGCGUUGUAUAACCUC
AAUAAUAUGGUUUGAGGGUGUCUACCAGGAACCGU
AAAUUCCUGAUUACAACGCAAACCGGAUGAUAGAC
CUCACCUCCCCGCCCAAUACUGAAAUCUCAUUAAU
ACGCAUACCCCCACUAUACACACGCAAUCACCACA
UUAGCACAAUGAAUAAUCAUCGUACGGGAGAAAAC
AUUCUAAACCCACAUGAGGAUCACCCAUGU.

Next, additional structures were tested in which the RNA-riboswitch sequence was inserted at alternative positions: (i) at the 5′ end of the RNA strand; and (ii) at position 109 thereof. The overall length of the designed constructs was maintained at about 240 nucleotides, and the length of the sequence between the RNA-riboswitch and the TR-RNA activator (also referred to as “intermediate spacer”) was adjusted accordingly, depending on the position of the riboswitch. The constructs were evaluated in both the NUPACK and OligoAnalyzer™ Tool platforms, at yeast growth conditions and at physiological human sera conditions. The sequences of the tested constructs, in which the sequences of the TR-RNA and riboswitch are marked as indicated above, are set forth below.

Construct containing the riboswitch at position 1
(5′ riboswitch RNA)-
(5′ riboswitch RNA, SEQ ID NO: 7)
CGCGUUGUAUAACCUCAAUAAUAUGGUUUGAGGGU
GUCUACCAGGAACCGUAAAUUCCUGAUUACAACGC
CCCCUGCGCUUCCGUCAAACCCCUGAAACCGGAUG
CAUAGACCUCACCUCCCCGCCCAAUACUGAAAUCU
AUUAAUACGCAUACCCCCACUAUACACACGCAAUC
ACCACAUUAGCACAAUGAAUAAUCAUCGUACGGGA
GAAAACAUUCUAAACCCACAUGAGGAUCACCCAUGU.
109-position riboswitch RNA-
(109-position riboswitch RNA, SEQ ID NO: 8)
CGCUUCCGUCAAACCCCUGAAACCGGAUGAUAGAC
CUCACCUCCCCGCCCAAUACUGAAAUCUCAUUAAU
ACGCAUACCCCCACUAUACACACGCAAUCACCACA
UUACGCGUUGUAUAACCUCAAUAAUAUGGUUUGAG
GGUGUCUACCAGGAACCGUAAAUUCCUGAUUACAA
CGCGCACAAUGAAUAAUCAUCGUACGGGAGAAAAC
AUUCUAAACCCACAUGAGGAUCACCCAUGU.

FIG. 10B depicts the analysis of the 5′ riboswitch RNA construct, and FIG. 10C depicts the analysis of the 109-position riboswitch RNA construct. As can be seen, the 5′ riboswitch RNA construct formed both functional RNA formations (marked by surrounding boxes, left-box—riboswitch, right-box—TR-RNA activator), as shown above with respect to the 19-position riboswitch RNA construct. However, the 109-position riboswitch RNA construct formed only the TR-RNA activator (marked by surrounding box), and not the functional riboswitch conformation. These results indicate that the formation of a functional riboswitch element is dependent on its location within the RNA construct. In particular, the results show that the 109-position riboswitch RNA construct, which was characterized by a shortened intermediate spacer of 43 ribonucleotides (nt) and an elongated 5′ spacer of 108 nt, did not enable the formation of a functional RNA that would provide the riboswitch activity under the relevant experimental conditions, while the other two constructs, characterized by a 5′ spacer of up to 18 nt in length and an intermediate spacer of 132-153 nt in length, were functional.

Accordingly, constructs containing the riboswitch element at their 5′ end were chosen for further analysis. In view of the effect of spacers as derived from the analysis above, it was tested whether factors other than the length of the spacer, such as its sequence, its relative abundance of G and C nucleotides in the spacer sequence (referred to as “GC content”) and specific structural elements, may affect the formation of functional riboswitch and TR-RNA elements.

For analyzing the effect of the GC content of the spacer, several constructs were designed, with the RNA-riboswitch positioned at the 5′-end and the TR-RNA activator positioned at the 3′-end separated by randomly-generated intermediate spacers, in which the GC content of the spacer sequence was 20%, 30%, 40%, 50%, 60%, 70%, or 80%. The sequences were evaluated by the OligoAnalyzer™ Tool. Exemplary sequences of the tested constructs are set forth below, with the TR-RNA underlined and the riboswitch marked in bold and underlined.

(20% GC-content, SEQ ID NO 9)
CGCGUUGUAUAACCUCAAUAAUAUGGUUUGAGGGU
GUCUACCAGGAACCGUAAAUUCCUGAUUACAACGC
UUUCAGUUAGUUAAUGUAUCUACUUUCUAAAUAUU
AUAUUUGUAUGUAUUUUUUGAUACUCAAAUGAAAU
UUGUAUUAUUAACUAAUAUAAGUUAGUAUCACAUU
AUAAGAAAGCAAAAACAAUUCCUGUAUAGGUAAGA
AAUUGCCAAAAAAACAUGAGGAUCACCCAUGU.
(30% GC-content-first alternative, SEQ ID NO 10)
CGCGUUGUAUAACCUCAAUAAUAUGGUUUGAGGGU
GUCUACCAGGAACCGUAAAUUCCUGAUUACAACGC
UCGUACUAGAUUUUUAGAUGAUUUUCAGAAUAAUU
GCGCUCUAUUAUAACUUAAUUAGUACCUAAAGGGU
GAAACGUCUAACUAUUGAACACCCUACGAUGUCUA
CAUAUGGGCAUAGUAAACCAGAAUCUCCCCCAAUC
GAGUGUAUAUUGUACAUGAGGAUCACCCAUGU.
(40% GC-content, SEQ ID NO 11)
CGCGUUGUAUAACCUCAAUAAUAUGGUUUGAGGGU
GUCUACCAGGAACCGUAAAUUCCUGAUUACAACGC
AAGAAGUUAUAGUGGUAAUUUUCCCAUGACAAGGA
CCCUAUGAACUUCAUAAGUCAAAUGUGUCAUCUUG
UUCAUUUGAUUGAAGGAUUUAAGACAAUAACGUCG
UGCUCGCGAUCAACUACCCAAAGACAUCAUUGAGC
GGGGCAAUUUUGGACAUGAGGAUCACCCAUGU.
(50% GC-content, SEQ ID NO 12)
CGCGUUGUAUAACCUCAAUAAUAUGGUUUGAGGGU
GUCUACCAGGAACCGUAAAUUCCUGAUUACAACGC
CGCCGGAACCGUGCUAUUGUGAAAUAAACACACGU
UCGGGAAACAAGGGCACACAUAGCCCAGAUUCGCC
CACCCAACUGCCAGUGCCCGAGCUUACCAUAUUCG
UUCUGUAUGCAGUGCUGUAAUUGCGUGCACGGAAG
AUUCAUUCUUGCGACAUGAGGAUCACCCAUGU.
(60% GC-content-first alternative, SEQ ID NO 13)
CGCGUUGUAUAACCUCAAUAAUAUGGUUUGAGGGU
GUCUACCAGGAACCGUAAAUUCCUGAUUACAACGC
CGAGGAAAUGCACACGGAGCUUCCCACAUACGCCG
CCCGCUUACGGCCGUCCGUUCCGACUCUAGGACAC
GUAAAACUCACCCUUACGCUCACAGCAGUGAAUCG
GGGUAGCAGGCCCCCGACCGUCCACUCGCCCGAGC
GAGCGGAUGAUGAACAUGAGGAUCACCCAUGU.
(70% GC-content-first alternative, SEQ ID NO 14)
CGCGUUGUAUAACCUCAAUAAUAUGGUUUGAGGGU
GUCUACCAGGAACCGUAAAUUCCUGAUUACAACGC
GAUGCCAUGAGCCACGAGGGCGAGUACGAGCUGGU
GGGCCCUGGCACUUAACCUGAACUAGGCGGCAACU
UUACGACGAGAGUUCGUGACUGGCCACGCGGAACG
CACUCCCCGUACGGAGCCUGCGCAUCAUGUCGGAA
GCGGGGUGGGCUGACAUGAGGAUCACCCAUGU.
(70% GC-content-second alternative, SEQ ID NO 15)
CGCGUUGUAUAACCUCAAUAAUAUGGUUUGAGGGU
GUCUACCAGGAACCGUAAAUUCCUGAUUACAACGC
CGUAAGCGAUCCCGGCCACACUGUCGGGAGGCCGC
CUCUGCCUCCGGCUUGGCGGCCACCCAGGUAUGGA
GGGUUGCCUGCCGGCUGCGUUCGCGCCAUCGCUAC
GCGGGCAGCGGGAGGCAACGAGCCGACGUACGCCC
GGCUAGGAAUCCCACAUGAGGAUCACCCAUGU.
(80% GC-content, SEQ ID NO 16)
CGCGUUGUAUAACCUCAAUAAUAUGGUUUGAGGGU
GUCUACCAGGAACCGUAAAUUCCUGAUUACAACGC
AAGCCGACGCUGCAGUCCGGCGCCGGGAGUGGCGG
CCCGCCCCCCACCAGUCCCUGGGCGCCGCGGUGGC
CUCAUACGGAGCCGGCCUGGAGCCGGCGGGGGGCG
CUGCGCCAGCGCUGGCGCGUUGGGGACCGGGCCGC
GAUCGUGCGCGGCACAUGAGGAUCACCCAUGU.

FIGS. 10D-10E present exemplary conformational evaluations of the RNA constructs with different levels of GC-content of the spacer, as described above. Specifically, FIG. 10D depicts the evaluation of the 40% GC-content construct (SEQ ID NO: 11), and FIG. 10E depicts the first alternative 70% GC-content construct (SEQ ID NO: 14), as will be described in further detail below.

As can be seen in FIG. 10D, both the riboswitch and the TR-RNA assumed their respective functional formations in the 40% GC-content construct under human sera conditions. Similar results were obtained on the constructs including 20%, 50%, and 60% GC-content spacers, in which the riboswitch and the TR-RNA assumed their respective functional formations. It is noted that while the analysis of the construct characterized by 30% GC content indicated that three other structures may be more likely to be formed under the tested conditions, the functional conformations were still determined to be formed with adequately high probability.

As can be seen in FIG. 10E, however, the functional TR-RNA activator structure was not formed in the structural analyses of the first alternative 70% GC-content construct (the functional riboswitch structure was formed; marked). Accordingly, a second alternative construct characterized by a 70% GC-content was designed and evaluated. The analyses indicated that both functional RNA structures were formed in this construct. Additional analyses revealed that the construct characterized by an 80% GC-content had a significantly lower probability of producing the functional RNA-riboswitch and the TR-RNA structures. In particular, the report indicated that six other structures were more likely to be formed under the tested conditions than the desired functional RNA structures.

In summary, spacers characterized by a GC content of 20% to 60% were found to facilitate the formation of functional RNA conformations in the tested constructs, whereas higher GC contents of 70% and 80% GC were associated with substantially reduced probability to form these conformations.

As the first alternative constructs characterized by 70% GC-content failed to form the functional TR-RNA activator structure, it was tested whether this failure may be associated with the formation of interfering structures at the vicinity of the TR-RNA. To this end, the 6-nucleotide sequence immediately adjacent (5′) to the TR-RNA at the first alternative 70% GC-content construct (GGGCUG), was inserted instead of the corresponding 6 nucleotides in the first alternative 30% GC-content construct. The same was done with a longer sequence containing the 12 nucleotides immediately adjacent 5′ to the TR-RNA (CGGGGUGGGCUG, SEQ ID NO: 18), which were inserted in place of the corresponding 12 nucleotides in the first alternative 30% GC-content construct and in the first alternative 60% GC-content construct. The resultant constructs (second and third alternative 30% constructs and second alternative 60% construct, SEQ ID NOs: 19-21, respectively) were evaluated using the OligoAnalyzer™ Tool. The sequences of the tested constructs are set forth below, in which the TR-RNA is underlined, the riboswitch is marked in bold and underlined and the 6-nt or 12-nt oligonucleotides italicized.

(second alternative 30% GC-content, SEQ ID NO 19)
CGCGUUGUAUAACCUCAAUAAUAUGGUUUGAGGGU
GUCUACCAGGAACCGUAAAUUCCUGAUUACAACGC
UCGUACUAGAUUUUUAGAUGAUUUUCAGAAUAAUU
GCGCUCUAUUAUAACUUAAUUAGUACCUAAAGGGU
GAAACGUCUAACUAUUGAACACCCUACGAUGUCUA
CAUAUGGGCAUAGUAAACCAGAAUCUCCCCCAAUC
GAGUGUAUGGGCUGACAUGAGGAUCACCCAUGU.
(third alternative 30% GC-content, SEQ ID NO 20)
CGCGUUGUAUAACCUCAAUAAUAUGGUUUGAGGGU
GUCUACCAGGAACCGUAAAUUCCUGAUUACAACGC
UCGUACUAGAUUUUUAGAUGAUUUUCAGAAUAAUU
GCGCUCUAUUAUAACUUAAUUAGUACCUAAAGGGU
GAAACGUCUAACUAUUGAACACCCUACGAUGUCUA
CAUAUGGGCAUAGUAAACCAGAAUCUCCCCCAAUC
CGGGGUGGGCUGACAUGAGGAUCACCCAUGU.
(second alternative 60% GC-content, SEQ ID NO 21)
CGCGUUGUAUAACCUCAAUAAUAUGGUUUGAGGGU
GUCUACCAGGAACCGUAAAUUCCUGAUUACAACGC
CGAGGAAAUGCACACGGAGCUUCCCACAUACGCCG
CCCGCUUACGGCCGUCCGUUCCGACUCUAGGACAC
GUAAAACUCACCCUUACGCUCACAGCAGUGAAUCG
GGGUAGCAGGCCCCCGACCGUCCACUCGCCCGAGC
CCGGGGUGGGCUGACAUGAGGAUCACCCAUGU.

In the evaluation results of all three constructs, both RNA-riboswitch and the TR-RNA activator appeared in their functional conformations. This indicates that the specific nucleotide sequence of the spacer, including in particular the sequence adjacent to the TR-RNA activator, does not on its own impair the formation of the TR-RNA functional structure.

Finally, in order to test whether the functional RNA construct of the adenine riboswitch can serve as a basis for delivery of additional functional riboswitches for applications in vitro and in vivo, a construct comprising an alternative riboswitch RNA sequence, namely cobalamin riboswitch (SEQ ID NO: 22), was designed and evaluated. First, the cobalamin riboswitch sequence was analyzed independently by the OligoAnalyzer™ Tool at human physiological conditions. The tested sequence is set forth below:

(Cobalamin riboswitch, SEQ ID NO: 22)
CCACUUGCCGGUCCUGUGAGUUAAUAGGGAAUCCAGUGCGAAUCUGGAGC
UGACGCGCAGCGGUAAGGAA.

FIG. 10F shows the structural evaluation of the cobalamin riboswitch, displaying the functional three-armed conformation associated with its functional unbound conformation.

Then the sequence of the entire riboswitch-RNA construct, including the cobalamin riboswitch, was evaluated. The RNA-riboswitch construct included the cobalamin riboswitch inserted at the 5′-end (bold and underlined), the TR-RNA activator positioned at the 3′-end (underlined), and a randomly-generated intermediate spacer sequence of 153 nt in length having a GC-content of 50%.

The sequence of the tested construct is set forth below.

(Cobalamin RNA-riboswitch construct,
SEQ ID NO: 23)
CCACUUGCCGGUCCUGUGAGUUAAUAGGGAAUCCA
GUGCGAAUCUGGAGCUGACGCGCAGCGGUAAGGAA
GAGCGCGGUGUGGCUGGCGAGCCUACCGUGUUGGA
UAAAGGCACAGGUUUUGGUUUAGGGGCGACCGUCA
CCUUUGUACCCCUAUUACCGAAGCGCUCGGAGCCU
AGCUGAACACUUGUUCAGAAAAAGCCGAGAUAAAA
CUGGCGGCACGGAACAUGAGGAUCACCCAUGU.

As can be seen in FIG. 10G, the functional conformations of both the cobalamin riboswitch and the TR-RNA activator were formed (marked by surrounding boxes, left-box—cobalamin RNA-riboswitch, right-box—TR-RNA activator).

Thus, the results show that the technology disclosed herein can be used with additional elements characterized by high structural complexity, including riboswitches other than adenine-riboswitch.

In summary, the results presented herein demonstrate the development of a nano-carrier system, comprising a functional RNA encapsulated inside a VLP. In particular, the formation of a system comprising functional RNAs containing a functional riboswitch element and a TR-RNA packaging sequence, encapsulated in a VLP formed of wild-type MS2 CP dimers, is described and characterized. The system enabled the chelation of excess metabolites and inhibition of their accumulation at toxic levels and formation of metabolite assemblies and amyloids in various environments including in living cells.

The ability to produce various functional RNA constructs retaining the secondary and/or tertiary structures associated with the above-discussed functional properties is further disclosed herein. In particular, certain structural properties of the RNA construct were demonstrated to be advantageous in maintaining RNA functionality.

For example, advantageous constructs were produced in which the TR-RNA is located at the 3′ end, the riboswitch is located at the 5′ end or preceded by a short oligonucleotide 5′ spacer (e.g. about 20 nt), with intermediate spacers longer than about 100-nt therebetween. The intermediate spacers included randomly generated sequences, with the length and GC content appearing to contribute to the role of the spacer in facilitating the functionality of the construct rather than the presence or absence of other specific interfering structures or sequences. For example, it was found that in constructs containing an intermediate spacer with a GC content higher than 60% (for example 70% or 80%) or shorter than about 50 nt, the probability of achieving functional RNA formations was substantially reduced or completely abolished, indicative of a correspondingly reduced effectivity of VLP formation and ligand-binding, which are required for the metabolite salvage effect in vitro and in vivo.

### Example 6. In Vivo Evaluation in an Animal Model

Following the successful rescue effect of the VLP-riboswitch nano-carriers on the mutant yeast model, riboswitch-VLPs are tested for treating an induced gout-disease mouse model. The induced gout-disease mouse model involves feeding the mice with a high-fat diet for several weeks. Consumption of this diet diminishes uric acid secretion and is associated with the development of high uric acid blood concentration (hyperuricemia) and the formation of monosodium urate monohydrate crystals which accumulate in the mouse paws, imitating gout disease.

A treatment model for examining the efficacy of VLP nano-carriers encapsulating a functional xanthin riboswitch RNA construct is conducted in mice. A double-expression plasmid is synthesized, as described in Example 1 and the “Materials and Methods” section above, where the functional RNA sequence of the double expression plasmid is selected from any one of the 5′ riboswitch RNA, 19-position riboswitch RNA, and 40%-GC content sequences, and the RNA-riboswitch encoding sequence encodes for xanthin riboswitch I. The sequence of the xanthin riboswitch use is as follows:

(SEQ ID NO: 24)
GGAGAGUGAGUAGAAGCGGUCAGUGCAGGCAGCUGCGCGGGACAUUGCUC
AGCAA.

The plasmid is introduced into bacterial hosts, and the VLP-riboswitch nano-carriers are self-assembled and purified therefrom, as also explained in Example 1 and in “Materials and Methods”. 28 specific pathogen-free (SPF) male C57BL/6 mice (male, 4-weeks-old) are randomly divided into four groups (n=7 mice/group): (1) control group (CT), fed normal diet and injected with PBS solution; (2) hyperuricemia model group (HFD), fed high-fat diet (10% yeast extract) and injected with PBS solution; (3) control+VLPs group (CT+VLPs), fed normal diet and periodically injected VLPs; and (4) hyperuricemia model+VLPs group (HFD+VLPs), fed high-fat diet (10% yeast extract) and injected with VLPs.

After completion of the treatment period (42 days), samples are collected at 48 h after the last injection of the VLPs. Blood samples are obtained from the eye socket vein of each mouse and centrifuged at 1300 g for 10 min at 4° C. Stool samples are removed from the colon and stored at −80° C. for further analysis. Foot joints tissue is also harvested from exsanguinated mice, flushed with 1×PBS, dissected longitudinally, and fixed in 4.0% formaldehyde overnight and decalcified in EDTA decalcification solution. The tissues are then embedded in paraffin. Sections of 5 m are cut from paraffin-embedded tissues and stained with hematoxylin and eosin (H&E) to evaluate the morphological changes and inflammation levels in the foot joints.

Foot pain is quantified by measuring the mechanical withdrawal threshold (MWT). MWT is assessed using the von Frey hairs (Stoelting, Wood Dale, IL) with a bending force ranging from 0.16 g to 26 g. In a quiet, temperature-controlled room, mice are individually placed in testing chambers with metal mesh floors at least 30 min before testing for acclimation. The number of stimulations for each force is five pokes. Sudden paw withdrawal, flinching, and paw licking are regarded as positive responses, while no response is considered as negative response. The minimum force that provokes at least three withdrawal responses of the right hind paw is defined as the WMT. All behavioral tests are performed by an investigator who is blinded to the experimental design.

Serum levels of cytokines are measured by a commercial multiplex mouse cytokine magnetic bead-based immunoassay (Bio-Plex Pro Mouse Cytokine 23-plex Assay, Bio-Rad Laboratories) according to the manufacturer's instructions. The cytokine screen includes IL-1α, IL-10, IL-2, IL-3, IL-4, IL-5, IL-6, IL-9, IL-10, IL-12p40, IL-12p70, IL-13, IL-17, Eotaxin, G-CSF, GM-CSF, IFN-γ, KC, MCP-1, MIP-1α, MIP-1β, RANTES and/or TNF-α. The mean fluorescence intensity from all the bead combinations tested is analyzed using the Bio-Plex system equipped with Bio-Plex Manager Software v6.0 (Bio-Rad Laboratories). In addition, serum uric acid is measured via an enzymatic-colorimetric method, using a standard test kit.

Upon thawing the foot joint or liver samples, the tissues (0.05 g of tissue per 1.0 ml buffer solution) are homogenized and centrifuged for 10 min at 12000 rpm at 4° C. The supernatants are evaluated for myeloperoxidase (MPO) activity in foot joint tissues and xanthine oxidase (XOD) and adenosine deaminase (ADA) activity in liver tissues, in accordance with the manufacturer's instructions (Jiancheng, Nanjing, China). Cytokines (IL-10, IL-6 and TNF-α) in the supernatants of foot joint is also detected using mouse ELISA commercial kits (CUSABIO, Wuhan, China) according to the manufacturer's instructions in the Varioskan Flash (Thermo scientific, America). The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-46. (canceled)

47. A nano-carrier system comprising a functional RNA encapsulated inside an Enterobacteria phage MS2 (MS2) virus-like particle (VLP), wherein the functional RNA comprises an RNA riboswitch and a packing sequence, and said riboswitch maintains its functionality while being encapsulated inside said VLP and is protected from nuclease degradation in vivo.

48. The nano-carrier system of claim 47, wherein said riboswitch is selected from the group consisting of a purine riboswitch, a cobalamin riboswitch, a fluoride riboswitch, a glutamine riboswitch, a glycine riboswitch and a xanthine riboswitch.

49. The nano-carrier system of claim 48, wherein said riboswitch is a naturally-occurring or synthetic adenine riboswitch, and/or wherein said packing sequence is an MS2 translational repression RNA (TR-RNA).

50. The nano-carrier system of claim 47, wherein said riboswitch is located 5′ to the TR-RNA, and said riboswitch and TR-RNA are separated by a non-coding RNA spacer characterized by a length of 100-200 nucleotides (nt) and a guanine-cytosine content (GC content) of 20%-70%, and wherein said riboswitch is located less than 30 nt from the 5′ end, said TR-RNA is located at the 3′ end, and the spacer is 120-170 nt in length and has a GC content of 40%-65%.

51. The nano-carrier system of claim 50, wherein said riboswitch is the pbuE A58U adenine riboswitch (SEQ ID NO: 1), or wherein said functional RNA has a nucleic acid sequence selected from the group consisting of SEQ ID NOs: 6, 7, 9-13, 19-21 and 23.

52. The nano-carrier system of claim 47, wherein said VLP comprises a capsid comprising wild-type (WT) MS2 coat proteins (CP).

53. The nano-carrier system of claim 52, wherein said WT MS2 CP is in the form of a single-chain CP dimer optionally comprising a linker between the CP monomer units, and said VLP is devoid of exogenously added targeting motifs.

54. The nano-carrier system of claim 47, comprising an adenine riboswitch encapsulated inside an Enterobacteria phage MS2 virus-like particle (VLP).

55. A nucleic acid construct encoding the nano-carrier system of claim 47.

56. A pharmaceutical composition comprising the nano-carrier system of claim 47 and a pharmaceutically acceptable carrier, excipient or diluent.

57. A method of treating a metabolic disorder characterized by elevated metabolite levels in a subject, or of inhibiting or delaying the formation of metabolite assemblies in said subject, the method comprising administering to the subject a pharmaceutical composition comprising a nano-carrier system, wherein the nano-carrier system comprises a virus-like particle (VLP) and a metabolite-specific functional RNA encapsulated inside the VLP, wherein the functional RNA maintains its ability to selectively bind the metabolite while being encapsulated inside said VLP, and is protected from nuclease degradation in vivo.

58. The method of claim 57, wherein said functional RNA comprises a riboswitch and a packing sequence, and/or wherein said VLP is an Enterobacteria phage MS2 (MS2) VLP

59. The method of claim 57 for treating a metabolic disorder characterized by elevated metabolite levels, wherein said metabolic disorder is selected from the group consisting of adenine phosphoribosyl transferase deficiency (APRT), adenosine deaminase deficiency (ADA deficiency), tyrosinemia and phenylketonuria, or wherein said subject is afflicted with hyperuricemia, Lesch-Nyhan syndrome and/or gout.

60. The method of claim 57 for inhibiting or delaying the formation of metabolite assemblies, wherein the metabolite is selected from the group consisting of adenine, tyrosine and phenylalanine, or wherein said subject is afflicted with, or is at risk for developing, kidney stones, gallstones, and/or cholesterol deposits

61. A method of reducing the levels of a metabolite in a food product, comprising contacting the food product or a precursor thereof with a chelating composition comprising a nano-carrier system, the nano-carrier system comprising a virus-like particle (VLP) and a metabolite-specific functional RNA encapsulated inside the VLP, wherein the functional RNA maintains its ability to selectively bind the metabolite while being encapsulated inside said VLP, and is protected from nuclease degradation in vivo.

62. The method of claim 61, wherein said metabolite is selected from the group consisting of lactic acid, ammonia and ionic forms thereof.

63. The method of claim 61, wherein said food product is a cultured meat product.

64. The method of claim 63, wherein said food product is selected from the group consisting of patties, sausages, burgers, meatballs, cutlets, and hotdogs.

65. The method of claim 61, wherein said VLP is an Enterobacteria phage MS2 (MS2) VLP.

66. A nano-carrier system comprising a virus-like particle (VLP) and a functional RNA encapsulated inside said VLP, wherein said VLP is configured to protect said functional RNA from nuclease degradation in vivo, and wherein said functional RNA maintains its functionality while being encapsulated inside said VLP.


SEQUENCE LISTING (24 sequences)

SEQ ID NO: 1 - DNA, 71 bp, synthetic construct
  source 1..71: mol_type = other DNA
gcgcgttgtataacctcaataatatggtttgagggtgtctaccaggaaccgtaaattcctgattacaacgc

SEQ ID NO: 2 - DNA, 19 bp, synthetic construct
  source 1..19: mol_type = other DNA
acatgaggatcacccatgt

SEQ ID NO: 3 - DNA, 240 bp, synthetic construct
  source 1..240: mol_type = other DNA
cgcttccgtcaaacccctgcgcgttgtataacctcaataatatggtttgagggtgtctaccaggaaccgtaaattcctgattacaacgcaaaccggatgatagacctcacctccccgcccaatactgaaatctcattaatacgcatacccccactatacacacgcaatcaccacattagcacaatgaataatcatcgtacgggagaaaacattctaaacccacatgaggatcacccatgt

SEQ ID NO: 4 - DNA, 834 bp, synthetic construct
  source 1..834: mol_type = other DNA
agccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacgctaactttactcagttcgttctcgtcgacaatggcggtacccatcaccatcaccatcacggtaccggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccgg

SEQ ID NO: 5 - DNA, 1739 bp, synthetic construct
  source 1..1739: mol_type = other DNA
acgactcactataggggaattgtgagcggataacaattcccctgtagaaataattttgtttaactttaataaggagatataccatggtggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccagaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcctatgagatgcttacgaaggttcaccttcaagagtttcttcctatgagagccgtacgtcaggtcggtactaacatcaagttaaatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacgctaactt

SEQ ID NO: 6 - RNA, 240 nt, synthetic construct
  source 1..240: mol_type = other RNA
cgcttccgtcaaacccctgcgcgttgtataacctcaataatatggtttgagggtgtctaccaggaaccgtaaattcctgattacaacgcaaaccggatgatagacctcacctccccgcccaatactgaaatctcattaatacgcatacccccactatacacacgcaatcaccacattagcacaatgaataatcatcgtacgggagaaaacattctaaacccacatgaggatcacccatgt

SEQ ID NO: 7 - RNA, 246 nt, synthetic construct
  source 1..246: mol_type = other RNA
cgcgttgtataacctcaataatatggtttgagggtgtctaccaggaaccgtaaattcctgattacaacgccccctgcgcttccgtcaaacccctgaaaccggatgatagacctcacctccccgcccaatactgaaatctcattaatacgcatacccccactatacacacgcaatcaccacattagcacaatgaataatcatcgtacgggagaaaacattctaaacccacatgaggatcacccatgt

SEQ ID NO: 8 - RNA, 240 nt, synthetic construct
  source 1..240: mol_type = other RNA
cgcttccgtcaaacccctgaaaccggatgatagacctcacctccccgcccaatactgaaatctcattaatacgcatacccccactatacacacgcaatcaccacattacgcgttgtataacctcaataatatggtttgagggtgtctaccaggaaccgtaaattcctgattacaacgcgcacaatgaataatcatcgtacgggagaaaacattctaaacccacatgaggatcacccatgt

SEQ ID NO: 9 - RNA, 242 nt, synthetic construct
  source 1..242: mol_type = other RNA
cgcgttgtataacctcaataatatggtttgagggtgtctaccaggaaccgtaaattcctgattacaacgctttcagttagttaatgtatctactttctaaatattatatttgtatgtattttttgatactcaaatgaaatttgtattattaactaatataagttagtatcacattataagaaagcaaaaacaattcctgtataggtaagaaattgccaaaaaaacatgaggatcacccatgt

SEQ ID NO: 10 - RNA, 242 nt, synthetic construct
  source 1..242: mol_type = other RNA
cgcgttgtataacctcaataatatggtttgagggtgtctaccaggaaccgtaaattcctgattacaacgctcgtactagatttttagatgattttcagaataattgcgctctattataacttaattagtacctaaagggtgaaacgtctaactattgaacaccctacgatgtctacatatgggcatagtaaaccagaatctcccccaatcgagtgtatattgtacatgaggatcacccatgt

SEQ ID NO: 11 - RNA, 242 nt, synthetic construct
  source 1..242: mol_type = other RNA
cgcgttgtataacctcaataatatggtttgagggtgtctaccaggaaccgtaaattcctgattacaacgcaagaagttatagtggtaattttcccatgacaaggaccctatgaacttcataagtcaaatgtgtcatcttgttcatttgattgaaggatttaagacaataacgtcgtgctcgcgatcaactacccaaagacatcattgagcggggcaattttggacatgaggatcacccatgt

SEQ ID NO: 12 - RNA, 242 nt, synthetic construct
  source 1..242: mol_type = other RNA
cgcgttgtataacctcaataatatggtttgagggtgtctaccaggaaccgtaaattcctgattacaacgccgccggaaccgtgctattgtgaaataaacacacgttcgggaaacaagggcacacatagcccagattcgcccacccaactgccagtgcccgagcttaccatattcgttctgtatgcagtgctgtaattgcgtgcacggaagattcattcttgcgacatgaggatcacccatgt

SEQ ID NO: 13 - RNA, 242 nt, synthetic construct
  source 1..242: mol_type = other RNA
cgcgttgtataacctcaataatatggtttgagggtgtctaccaggaaccgtaaattcctgattacaacgccgaggaaatgcacacggagcttcccacatacgccgcccgcttacggccgtccgttccgactctaggacacgtaaaactcacccttacgctcacagcagtgaatcggggtagcaggcccccgaccgtccactcgcccgagcgagcggatgatgaacatgaggatcacccatgt

SEQ ID NO: 14 - RNA, 242 nt, synthetic construct
  source 1..242: mol_type = other RNA
cgcgttgtataacctcaataatatggtttgagggtgtctaccaggaaccgtaaattcctgattacaacgcgatgccatgagccacgagggcgagtacgagctggtgggccctggcacttaacctgaactaggcggcaactttacgacgagagttcgtgactggccacgcggaacgcactccccgtacggagcctgcgcatcatgtcggaagcggggtgggctgacatgaggatcacccatgt

SEQ ID NO: 15 - RNA, 242 nt, synthetic construct
  source 1..242: mol_type = other RNA
cgcgttgtataacctcaataatatggtttgagggtgtctaccaggaaccgtaaattcctgattacaacgccgtaagcgatcccggccacactgtcgggaggccgcctctgcctccggcttggcggccacccaggtatggagggttgcctgccggctgcgttcgcgccatcgctacgcgggcagcgggaggcaacgagccgacgtacgcccggctaggaatcccacatgaggatcacccatgt

SEQ ID NO: 16 - RNA, 242 nt, synthetic construct
  source 1..242: mol_type = other RNA
cgcgttgtataacctcaataatatggtttgagggtgtctaccaggaaccgtaaattcctgattacaacgcaagccgacgctgcagtccggcgccgggagtggcggcccgccccccaccagtccctgggcgccgcggtggcctcatacggagccggcctggagccggcggggggcgctgcgccagcgctggcgcgttggggaccgggccgcgatcgtgcgcggcacatgaggatcacccatgt

SEQ ID NO: 17 - protein, 130 aa, Emesvirus zinderi
MASNFTQFVLVDNGGTGDVTVAPSNFANGVAEWISSNSRSQAYKVTCSVRQSSAQNRKYTIKVEVPKVATQTVGGVQLPVAAWRSYLNMELTIPIFATNDDCALIVKAMQGLLKDGNPIPSAIAANSGIY

SEQ ID NO: 18 - RNA, 12 nt, synthetic construct
  source 1..12: mol_type = other RNA
cggggtgggctg

SEQ ID NO: 19 - RNA, 243 nt, synthetic construct
  source 1..243: mol_type = other RNA
cgcgttgtataacctcaataatatggtttgagggtgtctaccaggaaccgtaaattcctgattacaacgctcgtactagatttttagatgattttcagaataattgcgctctattataacttaattagtacctaaagggtgaaacgtctaactattgaacaccctacgatgtctacatatgggcatagtaaaccagaatctcccccaatcgagtgtatgggctgacatgaggatcacccatgt

SEQ ID NO: 20 - RNA, 241 nt, synthetic construct
  source 1..241: mol_type = other RNA
cgcgttgtataacctcaataatatggtttgagggtgtctaccaggaaccgtaaattcctgattacaacgctcgtactagatttttagatgattttcagaataattgcgctctattataacttaattagtacctaaagggtgaaacgtctaactattgaacaccctacgatgtctacatatgggcatagtaaaccagaatctcccccaatccggggtgggctgacatgaggatcacccatgt

SEQ ID NO: 21 - RNA, 242 nt, synthetic construct
  source 1..242: mol_type = other RNA
cgcgttgtataacctcaataatatggtttgagggtgtctaccaggaaccgtaaattcctgattacaacgccgaggaaatgcacacggagcttcccacatacgccgcccgcttacggccgtccgttccgactctaggacacgtaaaactcacccttacgctcacagcagtgaatcggggtagcaggcccccgaccgtccactcgcccgagcccggggtgggctgacatgaggatcacccatgt

SEQ ID NO: 22 - RNA, 70 nt, synthetic construct
  source 1..70: mol_type = other RNA
ccacttgccggtcctgtgagttaatagggaatccagtgcgaatctggagctgacgcgcagcggtaaggaa

SEQ ID NO: 23 - RNA, 242 nt, synthetic construct
  source 1..242: mol_type = other RNA
ccacttgccggtcctgtgagttaatagggaatccagtgcgaatctggagctgacgcgcagcggtaaggaagagcgcggtgtggctggcgagcctaccgtgttggataaaggcacaggttttggtttaggggcgaccgtcacctttgtacccctattaccgaagcgctcggagcctagctgaacacttgttcagaaaaagccgagataaaactggcggcacggaacatgaggatcacccatgt

SEQ ID NO: 24 - RNA, 55 nt, synthetic construct
  source 1..55: mol_type = other RNA
ggagagtgagtagaagcggtcagtgcaggcagctgcgcgggacattgctcagcaa